SEQUENCE LISTING (16 sequences)

SEQ ID NO: 1 - protein, 120 aa, Homo sapiens
DNEDEELGTEGSTSEKRSPMKRERSRSHDSASSSLSSKASGSALGGESSAQPTALPQGEHARSPQPRGPAEEGRAPGEKQRPRASQGPPSAISRHSPGPTPQPDCSLRTGQRSVQVSVTS

SEQ ID NO: 2 - DNA, 24 bp, Artificial Sequence
  Description of Artificial Sequence Synthetic primer
caaagaataacctgttggccctgc

SEQ ID NO: 3 - DNA, 24 bp, Artificial Sequence
  Description of Artificial Sequence Synthetic primer
gacatgcctgcgctctcatactta

SEQ ID NO: 4 - DNA, 22 bp, Artificial Sequence
  Description of Artificial Sequence Synthetic primer
cagaggaccgtcagtagctcaa

SEQ ID NO: 5 - DNA, 27 bp, Artificial Sequence
  Description of Artificial Sequence Synthetic primer
ggaagatatgaggtcctagttgtgaat

SEQ ID NO: 6 - DNA, 25 bp, Artificial Sequence
  Description of Artificial Sequence Synthetic primer
agagacatgtaccttgaccatcgtc

SEQ ID NO: 7 - DNA, 23 bp, Artificial Sequence
  Description of Artificial Sequence Synthetic primer
gtctggacctcgtgaccattact

SEQ ID NO: 8 - DNA, 24 bp, Artificial Sequence
  Description of Artificial Sequence Synthetic primer
ctcgtcgctaccgtcgtgacttcg

SEQ ID NO: 9 - DNA, 25 bp, Artificial Sequence
  Description of Artificial Sequence Synthetic primer
cagatgccggttcaggtactcagtc

SEQ ID NO: 10 - DNA, 19 bp, Artificial Sequence
  Description of Artificial Sequence Synthetic primer
gaaggtgaaggtcggagtc

SEQ ID NO: 11 - DNA, 20 bp, Artificial Sequence
  Description of Artificial Sequence Synthetic primer
gaagatggtgatgggatttc

SEQ ID NO: 12 - protein, 21 aa, Homo sapiens
KTTRFEEVLHNSIEASLRSNN

SEQ ID NO: 13 - protein, 17 aa, Homo sapiens
SEKRSPMKRERSRSHDS

SEQ ID NO: 14 - protein, 18 aa, Homo sapiens
RLELEDEWQFRLRDEFQT

SEQ ID NO: 15 - protein, 6 aa, Artificial Sequence
  Description of Artificial Sequence Synthetic 6xHis tag
HHHHHH

SEQ ID NO: 16 - protein, 5 aa, Artificial Sequence
  Description of Artificial Sequence Synthetic peptide
GGGGS

(19) United States
(12) Patent Application Publication
(10) Pub. No.: US 2011/0123441 A1
(43) Pub. Date: May 26, 2011

(54) NOVEL GREB1A MONOCLONAL ANTIBODY

(75) Inventors: Marc E. Lippman, Coral Gables, FL (US); Harry James Hnatyszyn, Coral Gables, FL (US); Mingli Liu, Marietta, GA (US); James M. Rae, Dexter, MI (US)

(73) Assignees: UNIVERSITY OF MIAMI, Miami, FL (US); THE REGENTS OF THE UNIVERSITY OF MICHIGAN, Ann Arbor, MI (US)

(21) Appl. No.: 12/908,348

(22) Filed: Oct. 20, 2010

Related U.S. Application Data
(60) Provisional application No. 61/254,514, filed on Oct. 23, 2009.

(51) Int. Cl. A61K 51/00; A61K 39/395; A61K 39/00; C07K 14/00; A61P 35/00; C07K 16/18; C07H 21/04

(52) U.S. Cl. 424/1.49; 424/174.1; 424/178.1; 530/350; 530/387.9; 530/388.1; 530/389.1; 530/391.3; 536/23.1

(57) ABSTRACT

The generation and validation of a novel monoclonal GREB1 antibody (GREB1ab). Methods for the prognosis, diagnosis, assessment of disease progression, severity and outcome utilize GREB1 molecules as biomarkers. The GREB1 antibody is also a useful tool for investigations focused on the expression, distribution and function of GREB1 in normal and cancer tissues.

### CROSS REFERENCE TO RELATED APPLICATIONS

This application claims priority to U.S. provisional application No. 61/254,514, filed Oct. 23, 2009, which is incorporated herein by reference in its entirety.

### FIELD OF THE INVENTION

Embodiments of the invention comprise GREB1 binding molecules and biomarkers. Methods for the prognosis, diagnosis and assessment of disease severity enable the treatment of disease from the very early stages.

### BACKGROUND

Estrogen plays a central role in breast cancer development and progression. Increased lifetime exposure to estrogen correlates to several of the most potent risk factors for the development of breast cancer. Therapeutic agents that prevent estrogen action have been shown to be effective in preventing breast cancer. The physiological effects of estrogen are mediated by binding to specific steroid receptors, estrogen receptor alpha (ERα) and beta (ERβ). These estrogen receptors are regulators of hormone signaling and regulate gene expression via direct or indirect interaction with promoters. It is this involvement in the regulation of specific genes that establishes estrogen as a significant factor in breast cancer pathogenesis.

### SUMMARY

This Summary is provided to present a summary of the invention to briefly indicate the nature and substance of the invention. It is submitted with the understanding that it will not be used to interpret or limit the scope or meaning of the claims.

In a preferred embodiment, an antibody specifically binds to human GREB1. In some embodiments, the antibodies are monoclonal antibodies. For example, a GREB1 monoclonal antibody detects a 216 kD protein corresponding to GREB1a in ER⁺ breast cancer cells expressing GREB1 as well as cells transfected with a GREB1 expression plasmid. This antibody has been validated for use in Western blotting and immunohistochemical (IHC) detection of GREB1 in breast and prostate cancer tissue microarrays.

The antibody or antibodies described herein have applications for research use, such as, for example, Western blots, immunohistochemistry, flow cytometry, imaging, ELISA, as well as clinical applications for prognostics and potential monitoring of therapeutic regimens using, for example, automated antibody-based platforms and pathological analysis of tumor/tissue samples.

In another preferred embodiment, detection of a biomarker comprising GREB1, mutants, isoforms, variants, fragments, or combinations thereof is prognostic or diagnostic of a disease, for example, cancer.

Other aspects are described infra.

### BRIEF DESCRIPTION OF THE DRAWINGS

FIGS. 1A-1B show the correlation of GREB1 protein expression with mRNA levels in breast cancer cell lines. FIG. 1A is a graph showing GREB1 mRNA levels in ER⁺ and ER⁻ breast cancer cell lines as detected using RT-PCR. GAPDH expression levels are provided as a comparative control. FIG. 1B shows the corresponding Western blot analysis of ER⁺ and ER⁻ breast cancer cell lines using the GREB1ab. A single band of 216 kD corresponds to human GREB1.

FIGS. 2A-2B show the specificity of GREB1 antibody. FIG. 2A shows that Western blot analysis detects GREB1 protein expression in MCF-7 cells treated with estrogen for 24, 48 and 72 hours while no protein is detected in MCF-7 cells grown in estrogen-free conditions. GREB1 protein expression was reduced in the MCF-7 cells treated with estrogen plus ICI 182,780 compared to that of observed in cells treated with estrogen alone. FIG. 2B shows that there is a loss of detectable GREB1 protein when GREB1 is knocked down with target specific siRNA at 48 hours. Loss of GREB1 is notable as early as 24 hours and lasts up to 72 hours in a time course study. Blots were stripped and restained for β-actin as a comparative sample loading control.

FIGS. 3A-3B show the detection of GREB1 protein following delivery with an adenoviral vector. MDA-MB-231, MCF-7 cells and 3-day estrogen depleted MCF-7 cells were seeded onto plates, grown to 60% confluence, and infected with Ad-CMV-Null and Ad-GREB1 with 20 MOI. Twenty-four hours after infection, cells were collected and assayed for mRNA and protein expression. FIG. 3A is a graph showing that relative RNA levels determined by real-time RT-PCR for estrogen depleted ER⁺ MCF-7 cells and ER-MDA-MB-231 cells transduced with a control vector and adenoviral vector expressing GREB1. FIG. 3B is a blot showing that the cells transduced with Ad-GREB1 express high GREB1 protein levels, the exogenous GREB1 bands have a mobility identical to that of endogenous GREB1 induced by estradiol stimulation (lane 5 vs. lane 2 and 3; lane 12 vs. lane 10). GREB1 was undetectable in estrogen deprived MCF-7, MDA-MB-231 asynchronous cultures and MDA-MB-231 transduced with empty vector (lanes 1, 9, 8 and 4). Detectable expression of GREB1 in MCF-7 cells transduced with Ad-CMV-null, just as MCF-7 cells transduced Ad-GREB1 (lane 6 and lane 7) was due to the existence of endogenous hormone in serum. Blots were stripped and restained for β-actin as a comparative sample loading control.

FIG. 4 shows the immunohistochemical staining for GREB1 expression in breast cancer cell lines. Four breast cancer cell lines (ER⁺; MCF-7, Ly2: ER⁻; SUM225, MDA-MB 231) were stained with standard hematoxylin and eosin as well as immunohistochemically for GREB1 and ERα.

FIGS. 5A-5B shows the immunohistochemical staining of breast cancer tissues using the GREB1 antibody. FIG. 5A shows that GREB1 protein expression in breast cancer tissue sections from whole tumor blocks. GREB1 protein was detected in ERα positive breast cancer tissue as well as normal breast tissue with little GREB1 expression in ERα negative breast cancer tissue. FIG. 5B shows representative micrographs from two tumors included in the breast tissue microarrays. Panel B2 shows negative GREB1 staining in an ER-negative breast cancer, whereas panel C2 reveals GREB1 staining in the normal tissue adjacent to the B2 tumor sample. GREB1 protein was detected in both tumor (panel D7) and the uninvolved normal tissue paired with D (panel F7) in an ER-positive breast cancer.

FIGS. 6A-6C show the inverse correlation of GREB1 and HER2 protein expression in breast cancers. FIG. 6A shows representative micrographs of HER2 immunostaining 0 (−), 1+, 2+, and 3+, of breast cancer slides. FIG. 6B shows that trastuzumab (T) and lapatinib (L) increase the GREB1 mRNA expression by real time RT-PCR analysis. BT-474, a ER positive, HER2 amplified breast cancer cell line, was treated with trastuzumab and lapatinib, or the combination (T+L) for 12, 24, 36 and 48 hours. The pretreatment of BT-474 cells with trastuzumab increases GREB1 mRNA by 2.5 to 6.4 fold, with a maximum increase at 48 hours. Lapatinib enhances GREB1 mRNA expression by 2 to 10 fold by 48 hours. FIG. 6C shows that trastuzumab and lapatinib increase expression of IRS-1, IGFBP4 and bcl-2 mRNA as detected by real time RT-PCR analysis. BT-474 cells were treated with trastuzumab and lapatinib or the combination (T+L) for 12, 24, 36 and 48 hours, respectively

### DETAILED DESCRIPTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The term “antibody”, is inclusive of all species, including human and humanized antibodies and the antigenic target, for example, GREB1, can be from any species.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H-CH1} by a disulfide bond. The F(ab)′₂ may be reduced under mild conditions to break the disulfide linkage in the hinge region thereby converting the (Fab′)₂ dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Embodiments include single chain antibodies, single chain Fv (scFv) antibodies in which a variable heavy and a variable light chain are joined together (directly or through a peptide linker) to form a continuous polypeptide.

An “antigen-binding site” or “binding portion” refers to the part of an immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions” or “FRs”. Thus, the term “FR” refers to amino acid sequences that are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen binding “surface”. This surface mediates recognition and binding of the target antigen. The three hypervariable regions of each of the heavy and light chains are referred to as “complementarity determining regions” or “CDRs” and are characterized, for example by Kabat et al. Sequences of proteins of immunological interest, 4th ed. U.S. Dept. Health and Human Services, Public Health Services, Bethesda, Md. (1987).

As used herein, the terms “immunological binding” and “immunological binding properties” refer to the non-covalent interactions of the type which occur between an immunoglobulin molecule and an antigen for which the immunoglobulin is specific. The strength or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity and is thus equal to the dissociation constant Kd. See, generally, Davies et al. Ann. Rev. Biochem., 59: 439-473 (1990).

The phrase “specifically binds to a protein” or “specifically immunoreactive with”, when referring to an antibody refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to a protein under such conditions may require an antibody that is selected for its specificity for a particular protein. For example, GREB1 antibodies can be raised to the GREB1 protein that specifically bind to GREB1 and not to other proteins present in a tissue sample. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, or flow cytometry.

“Treating” or “treatment” of a state, disorder or condition includes: (1) Preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition; or (2) Inhibiting the state, disorder or condition, i.e., arresting, reducing or delaying the development of the disease or a relapse thereof (in case of maintenance treatment) or at least one clinical or sub-clinical symptom thereof; or (3) Relieving the disease, i.e., causing regression of the state, disorder or condition or at least one of its clinical or sub-clinical symptoms. The benefit to a subject to be treated is either statistically significant or at least perceptible to the patient or to the physician.

“Patient” or “subject” refers to mammals and includes human and veterinary subjects.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As defined herein, a “therapeutically effective” amount of a compound (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

### GREB1 Compositions

The human Gene Regulated by Estrogen in Breast cancer 1 (GREB1) gene is located on chromosome 2 (2p25.1), has at least three different noncoding 5′ exons, 6 probable alternative promoters and at least 10 potential splice variants. However, each transcript uses the same initiation codon and generates various splicing patterns that involve the 3′ end of the gene. The longest isoform, GREB1a, is 8482 bases (1949 aa) with the divergence of sequence homology from isoform b (457 aa) after nucleotide 1346 and isoform c (409 aa) after nucleotide 1159. In order to better understand estrogen-mediated promotion of breast cancer development as well as generate effective therapies, it is imperative to understand which genes are uniquely responsible for estrogen-stimulated growth. Using multiple estrogen-dependent breast cancer cell lines and microarray technology, the inventors herein, generated gene expression profiles to identify key genes involved in hormone-regulated growth. Genes with hormone-regulated expression profiles common to all cell lines and correlated to proliferation of the cells in response to estrogen or anti-estrogen treatment were documented.

GREB1 is an estrogen-regulated gene that mediates estrogen-stimulated cell proliferation and is a candidate clinical marker for response to endocrine therapy as well as potential therapeutic target. GREB1a mRNA expression in breast cancers are rapidly induced by estrogen (E2) and siRNA silencing of GREB1a mRNA blocks estrogen-induced growth in estrogen receptor (ER) positive cell lines. Without wishing to be bound by theory, it is thought that GREB1 plays a role in hormone-stimulated breast cancer proliferation and would be a candidate for clinical applications. To date, there is little evidence regarding GREB1 protein expression or function in normal and breast cancer cells. The lack of a specific antibody to GREB1 has further inhibited correlative protein-based investigations in breast tissues. A monoclonal antibody targeting GREB1 would serve as a useful tool for applications designed to verify RNA-based expression data as well as imaging methodologies to explore the localization of GREB1 as well as provide initial insight into the function of this protein in hormone-responsive tissues.

Briefly, the data described in detail in the Examples section which follows, show that using Northern blot analysis and isoform-specific RT-PCR in breast cancer studies, only the full-length isoform GREB1a is expressed in ER⁺ breast cancer cell lines with very low levels of the shorter isoforms. A hybridoma was generated (see, the Examples section which follows) expressing a murine monoclonal antibody (IgG1) against a 119 amino acid peptide (DNEDEELGTE GSTSEKRSPM KRERSRSHDS ASSSLSSKAS GSALGGESSA QPTALPQGEH ARSPQPRGPA EEGRAPGEKQ RPRASQGPPS AISRHSPGPTP QPDCSLRTGQ RSVQVSVTS (SEQ ID NO: 1)) specific to human GREB1 (GREB1ab). This GREB1ab detects a 216 kD protein corresponding to GREB1a in ER⁺ breast cancer cells expressing GREB1 as well as cells transfected with a GREB1 expression plasmid. This antibody was validated for use in Western blotting and immunohistochemical (IHC) detection of GREB1 in breast and prostate cancer tissue microarrays. As the role of GREB1 is being defined in hormone-mediated cancers, this antibody will have applications for research use (ex: Western blots, immunohistochemistry, flow cytometry, imaging, ELISA) as well as clinical applications for prognostics and potential monitoring of therapeutic regimens using automated antibody-based platforms and pathological analysis of tumor/tissue samples.

In a preferred embodiment, an antibody specifically binds to GREB1 proteins, peptides, variants, orthologs, alleles, isoforms, splice variants, derivatives or mutants thereof.

In another preferred embodiment, an antibody is a monoclonal antibody specific for GREB1 proteins, peptides, variants, orthologs, alleles, isoforms, splice variants, derivatives or mutants thereof.

In another preferred embodiment, an antibody specifically binds to the amino acid sequences set forth as SEQ ID NOS: 1, 12, 13 or 14.

In another preferred embodiment, an antibody specifically binds to amino acids having at least 50% sequence identity to the amino acid sequences set forth as SEQ ID NOS: 1, 12, 13 or 14.

In another preferred embodiment, the antibody comprises a label for detecting the antibody in vivo and to monitor the expression of GREB1 during therapy.

Radiolabeling: In another preferred embodiment, the antibody of the invention can be radiolabeled. Uses include therapeutic and imaging for diagnostic purposes. The label may be a radioactive atom, an enzyme, or a chromophore moiety. Methods for labeling antibodies have been described, for example, by Hunter and Greenwood, Nature, 144:945 (1962) and by David et al. Biochemistry 13:1014-1021 (1974). Additional methods for labeling antibodies have been described in U.S. Pat. Nos. 3,940,475 and 3,645,090. Methods for labeling oligonucleotide probes have been described, for example, by Leary et al. Proc. Natl. Acad. Sci. USA (1983) 80:4045; Renz and Kurz, Nucl. Acids Res. (1984) 12:3435; Richardson and Gumport, Nucl. Acids Res. (1983) 11:6167; Smith et al. Nucl. Acids Res. (1985) 13:2399; and Meinkoth and Wahl, Anal. Biochem. (1984) 138:267.

The label may be radioactive. Some examples of useful radioactive labels include ³²P, ¹²⁵I, ¹³¹ I, and ³H. Use of radioactive labels have been described in U.K. 2,034,323, U.S. Pat. No. 4,358,535, and U.S. Pat. No. 4,302,204.

Some examples of non-radioactive labels include enzymes, chromophores, atoms and molecules detectable by electron microscopy, and metal ions detectable by their magnetic properties.

Some useful enzymatic labels include enzymes that cause a detectable change in a substrate. Some useful enzymes and their substrates include, for example, horseradish peroxidase (pyrogallol and o-phenylenediamine), β-galactosidase (fluorescein β-D-galactopyranoside), and alkaline phosphatase (5-bromo-4-chloro-3-indolyl phosphate/nitro blue tetrazolium). The use of enzymatic labels has been described in U.K. 2,019,404, EP 63,879, and by Rotman, Proc. Natl. Acad. Sci. USA, 47, 1981-1991 (1961).

Useful chromophores include, for example, fluorescent, chemiluminescent, and bioluminescent molecules, as well as dyes. Some specific chromophores useful in the present invention include, for example, fluorescein, rhodamine, Texas red, phycoerythrin, umbelliferone, luminol.

The labels may be conjugated to the antibody or nucleotide probe by methods that are well known in the art. The labels may be directly attached through a functional group on the probe. The probe either contains or can be caused to contain such a functional group. Some examples of suitable functional groups include, for example, amino, carboxyl, sulfhydryl, maleimide, isocyanate, isothiocyanate. Alternatively, labels such as enzymes and chromophores may be conjugated to the antibodies or nucleotides by means of coupling agents, such as dialdehydes, carbodiimides, dimaleimides, and the like.

The label may also be conjugated to the probe by means of a ligand attached to the probe by a method described above and a receptor for that ligand attached to the label. Any of the known ligand-receptor combinations is suitable. Some suitable ligand-receptor pairs include, for example, biotin-avidin or biotin-streptavidin, and antibody-antigen.

In another preferred embodiment, the chimeric fusion molecules of the invention can be used for imaging. In imaging uses, the complexes are labeled so that they can be detected outside the body. Typical labels are radioisotopes, usually ones with short half-lives. The usual imaging radioisotopes, such as ¹²³I, ¹²⁴I, ¹²⁵I, ¹³¹I, ^{99m}TC, ¹⁸⁶Re, ¹⁸⁸Re, ⁶⁴Cu, ⁶⁷Cu, ²¹² Bi, ²¹³Bi, ⁶⁷Ga, ⁹⁰Y, ¹¹¹In, ¹⁸F, ³H, ¹⁴C, ³⁵S or ³²P can be used. Nuclear magnetic resonance (NMR) imaging enhancers, such as gadolinium-153, can also be used to label the complex for detection by NMR. Methods and reagents for performing the labeling, either in the polynucleotide or in the protein moiety, are considered known in the art.

GREB1 Specific Molecules: The antibody of the present invention may be a monoclonal antibody or a polyclonal antibody. The Examples section describes in detail the generation of antibodies which are specific for GREB1 molecules.

Other methods to develop GREB1 antibodies can also be utilized. For example, GREB1 polypeptide sequences are used to determine appropriate nucleic acid sequences encoding the GREB1 antibodies and the nucleic acids sequences then used to express one or more GREB1 antibodies. The nucleic acid sequence may be optimized to reflect particular codon “preferences” for various expression systems according to standard methods well known to those of skill in the art. Using the sequence information provided, the nucleic acids may be synthesized according to a number of standard methods known to those of skill in the art. Oligonucleotide synthesis, is preferably carried out on commercially available solid phase oligonucleotide synthesis machines (Needham-VanDevanter et al. (1984) Nucleic Acids Res. 12:6159-6168) or manually synthesized using the solid phase phosphoramidite triester method described by Beaucage et. al. (Beaucage et. al. (1981) Tetrahedron Letts. 22(20): 1859-1862).

Once a nucleic acid encoding a GREB1 antibody is synthesized it may be amplified and/or cloned according to standard methods. Molecular cloning techniques to achieve these ends are known in the art. A wide variety of cloning and in vitro amplification methods suitable for the construction of recombinant nucleic acids are known to persons of skill. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel). Methods of producing recombinant immunoglobulins are also known in the art. See, Cabilly, U.S. Pat. No. 4,816,567; and Queen et al. (1989) Proc. Nat'l Acad. Sci. USA 86: 10029-10033.

Examples of techniques sufficient to direct persons of skill through in vitro amplification methods, including the polymerase chain reaction (PCR) the ligase chain reaction (LCR), Qβ-replicase amplification and other RNA polymerase mediated techniques are found in Berger, Sambrook, and Ausubel, as well as Mullis et al., (1987) U.S. Pat. No. 4,683,202.

Once the nucleic acid for a GREB1 antibody is isolated and cloned, one may express the gene in a variety of recombinantly engineered cells known to those of skill in the art. Examples of such cells include bacteria, yeast, filamentous fungi, insect (especially employing baculoviral vectors), and mammalian cells. It is expected that those of skill in the art are knowledgeable in the numerous expression systems available for expression of GREB1 antibodies.

In brief, the expression of natural or synthetic nucleic acids encoding GREB1 antibodies will typically be achieved by operably linking a nucleic acid encoding the antibody to a promoter (which is either constitutive or inducible), and incorporating the construct into an expression vector. The vectors can be suitable for replication and integration in prokaryotes, eukaryotes, or both. Typical cloning vectors contain transcription and translation terminators, initiation sequences, and promoters useful for regulation of the expression of the nucleic acid encoding the GREB1 antibody. The vectors optionally comprise generic expression cassettes containing at least one independent terminator sequence, sequences permitting replication of the cassette in both eukaryotes and prokaryotes, i.e., shuttle vectors, and selection markers for both prokaryotic and eukaryotic systems.

To obtain high levels of expression of a cloned nucleic acid it is common to construct expression plasmids which typically contain a strong promoter to direct transcription, a ribosome binding site for translational initiation, and a transcription/translation terminator. Examples of regulatory regions suitable for this purpose in E. coli are the promoter and operator region of the E. coli tryptophan biosynthetic pathway as described by Yanofsky, (1984) J Bacteriol., 158:1018-1024 and the leftward promoter of phage lambda (P_{E}) as described by Herskowitz and Hagen (1980) Ann. Rev. Genet., 14:399-445. The inclusion of selection markers in DNA vectors transformed in E. coli is also useful. Examples of such markers include genes specifying resistance to ampicillin, tetracycline, or chloramphenicol. See Sambrook for details concerning selection markers, e.g., for use in E. coli. The GREB1 antibodies produced by prokaryotic cells may require exposure to chaotropic agents for proper folding. During purification from, e.g., E. coli, the expressed protein is optionally denatured and then renatured. This is accomplished, e.g., by solubilizing the bacterially produced antibodies in a chaotropic agent such as guanidine HCl. The antibody is then renatured, either by slow dialysis or by gel filtration. See, U.S. Pat. No. 4,511,503.

Methods of transfecting and expressing genes in mammalian cells are known in the art. Transducing cells with nucleic acids can involve, for example, incubating viral vectors containing GREB1 nucleic acids with cells within the host range of the vector.

The culture of cells used in the present invention, including cell lines and cultured cells from tissue or blood samples is well known in the art (see, e.g., Freshney (1994) Culture of Animal Cells, a Manual of Basic Technique, third edition, Wiley-Liss, N.Y. and the references cited therein).

GREB1 antibodies of this invention include individual, allelic, strain, or species variants, and fragments thereof, both in their naturally occurring (full-length) forms and in recombinant forms. The antibodies can be raised in their native configurations or in non-native configurations. Anti-idiotypic antibodies can also be generated.

Other examples of methods for making antibodies that specifically bind to a particular epitope are known to persons of skill. The following discussion is presented as a general overview of the techniques available; however, one of skill will recognize that many variations upon the following methods are known.

If a polyclonal antibody, for example, is to be produced, the GREB1 polypeptide is bound to a carrier protein such as BSA (bovine serum albumin), porcine thyroid globulin, or KLH (keyhole limpet hemocyanin) using an appropriate condensing agent such as carbodiimide or maleimide to produce an antigen for immunization (immunogen). The binding of the antigenic polypeptide to the carrier protein here may be carried out by an ordinal method known in this art. For example, KLH used as a carrier protein is maleimidated to bind the antigenic polypeptide. In this method, KLH is maleimidated by reacting with, preferably, a bifunctional condensing agent such as Sulfo-SMCC (sulfosuccimidyl 4-(N-maleimidomethyl)cyclohexane-1-carboxylate), followed by reaction with the antigenic polypeptide in which cysteine is added to one end desired for binding, the amino end or the carboxyl end of the peptide. As a result, the maleimidated KLH can readily bind to the antigenic polypeptide through thiol and thereby, an antigen for immunization is prepared. Alternatively, if carbodiimide is used, the KLH and the polypeptide can be bound together by forming a peptide bond with dehydration condensation between KLH and the antigenic polypeptide.

A solution containing the immunogen prepared as described above is mixed with an adjuvant, if necessary, and an animal generally used for producing an antibody (e.g. mouse, rat, rabbit, guinea pig, sheep, or goat) is subcutaneously or intraperitoneally immunized with the mixture repeatedly every 2 to 3 weeks. Blood is taken from the immunized animal and serum is separated therefrom to obtain antiserum. Methods of purifying an antibody include: a method where serum is heat-treated to inactivate the complement, followed by salting-out using ammonium sulfate; a method of purifying an immunoglobulin fraction by, for example, ion exchange chromatography; and a method of purifying an antibody by affinity column chromatography using a column on which a certain polypeptide is immobilized. Of those, the method using affinity column chromatography is preferable. Here, as a polypeptide for purification that is immobilized on a column (hereinafter, which is also referred to as a “polypeptide for purification”), a polypeptide having the same sequence or the sequence of a portion thereof may be selected depending on the amino acid sequence of the antigenic polypeptide used for immunization.

Also according to this aspect of the invention, in some embodiments the method further entails administering to the subject an adjuvant. An “adjuvant” as used herein refers to an antigen-nonspecific stimulator of the immune response. Adjuvants induce a strong antibody response to soluble antigens (Harlow and Lane, Antibodies: A Laboratory Manual, Cold Spring Harbor, N.Y. Current Edition; hereby incorporated by reference). The overall effect of adjuvants is dramatic and their importance cannot be overemphasized. The action of an adjuvant allows much smaller doses of antigen to be used and generates antibody responses that are more persistent. The nonspecific activation of the immune response often can spell the difference between success and failure in obtaining an immune response. Adjuvants should be used for first injections unless there is some very specific reason to avoid this. Most adjuvants incorporate two components. One component is designed to protect the antigen from rapid catabolism (e.g., liposomes or synthetic surfactants (Hunter et al. 1981)). Liposomes are only effective when the immunogen is incorporated into the outer lipid layer; entrapped molecules are not seen by the immune system. The other component is a substance that will stimulate the immune response nonspecifically. These substances act by raising the level of lymphokines Lymphokines stimulate the activity of antigen-processing cells directly and cause a local inflammatory reaction at the site of injection. LPS is reasonably toxic, and, through analysis of its structural components, most of its properties as an adjuvant have been shown to be in a portion known as lipid A. Lipid A is available in a number of synthetic and natural forms that are much less toxic than LPS but still retain most of the better adjuvant properties of parental LPS molecule. Lipid A compounds are often delivered using liposomes.

Adjuvants include, but are not limited to, alum (e.g., aluminum hydroxide, aluminum phosphate); saponins purified from the bark of the Q. saponaria tree, such as QS21 (a glycolipid that elutes in the 21^{st} peak with HPLC fractionation; Aquila Biopharmaceuticals, Inc., Worcester, Mass.); poly[di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.), emulsion-based formulations including mineral oil, non-mineral oil, water-in-oil or oil-in-water-in oil emulsion, oil-in-water emulsions such as Seppic ISA series of Montanide adjuvants; and PROVAX, ISCOMs (Immunostimulating complexes which contain mixed saponins, lipids and form virus-sized particles with pores that can hold antigen; SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxpropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

In some instances, it is desirable to prepare monoclonal antibodies from various mammalian hosts, such as mice, rodents, primates, humans, etc and avian, for example, chickens. Descriptions of techniques for preparing such monoclonal antibodies are found in, e.g., Stites et al. (eds.) Basic and Clinical Immunology (4th ed.) Lange Medical Publications, Los Altos, Calif., and references cited therein; Harlow and Lane, supra; Goding (1986) Monoclonal Antibodies: Principles and Practice (2d ed.) Academic Press, New York, N.Y.; and Kohler and Milstein (1975) Nature 256: 495-497.

Phage Display can be Used to Increase Antibody Affinity: To create higher affinity antibodies, mutant scFv gene repertories, based on the sequence of a binding scFv (e.g., SEQ ID NOS: 1, 12-14), are created and expressed on the surface of phage. Display of antibody fragments on the surface of viruses which infect bacteria (bacteriophage or phage) makes it possible to produce human or other mammalian antibodies (e.g. scFvs) with a wide range of affinities and kinetic characteristics. To display antibody fragments on the surface of phage (phage display), an antibody fragment gene is inserted into the gene encoding a phage surface protein (e.g., pIII) and the antibody fragment-pIII fusion protein is expressed on the phage surface (McCafferty et al. (1990) Nature, 348: 552-554; Hoogenboom et al. (1991) Nucleic Acids Res., 19: 4133-4137).

Since the antibody fragments on the surface of the phage are functional, those phage bearing antigen binding antibody fragments can be separated from non-binding or lower affinity phage by antigen affinity chromatography (McCafferty et al. (1990) Nature, 348: 552-554). Mixtures of phage are allowed to bind to the affinity matrix, non-binding or lower affinity phage are removed by washing, and bound phage are eluted by treatment with acid or alkali. Depending on the affinity of the antibody fragment, enrichment factors of 20 fold-1,000,000 fold are obtained by single round of affinity selection.

By infecting bacteria with the eluted phage or modified variants of the eluted phage as described below, more phage can be grown and subjected to another round of selection. In this way, an enrichment of 1000 fold in one round becomes 1,000,000 fold in two rounds of selection (McCafferty et al. (1990) Nature, 348: 552-554). Thus, even when enrichments in each round are low, multiple rounds of affinity selection leads to the isolation of rare phage and the genetic material contained within which encodes the sequence of the binding antibody (Marks et al. (1991) J. Mol. Biol., 222: 581-597). The physical link between genotype and phenotype provided by phage display makes it possible to test every member of an antibody fragment library for binding to antigen, even with libraries as large as 100,000,000 clones.

One approach for creating mutant scFv gene repertoires involves replacing either the V_{H} or V_{L} gene from a binding scFv with a repertoire of V_{H} or V_{L} genes (chain shuffling) (Clackson et al. (1991) Nature, 352: 624-628). Such gene repertoires contain numerous variable genes derived from the same germline gene as the binding scFv, but with point mutations (Marks et al. (1992) Bio/Technology, 10: 779-783). Using light or heavy chain shuffling and phage display, the binding avidities of GREB1 antibody fragment can be dramatically increased (see, e.g., Marks et al. (1992) Bio/Technology, 10: 779-785 in which the affinity of a human scFv antibody fragment which bound the hapten phenyloxazolone (phox) was increased from 300 nM to 15 nM (20 fold)).

Thus, to alter the affinity of GREB1 antibody a mutant scFv gene repertoire is created containing the V_{H} gene of the GREB1 antibody and a V_{L} gene repertoire (light chain shuffling). Alternatively, an scFv gene repertoire is created containing the V_{L} gene of a known GREB1 antibody and a V_{H} gene repertoire (heavy chain shuffling). The scFv gene repertoire is cloned into a phage display vector (e.g., pHEN-1, Hoogenboom et al. (1991) Nucleic Acids Res., 19: 4133-4137) and after transformation a library of transformants is obtained. The antigen concentration is decreased in each round of selection, reaching a concentration less than the desired Kd by the final rounds of selection. This results in the selection of phage on the basis of affinity (Hawkins et al. (1992) J. Mol. Biol. 226: 889-896).

Another method to increase the affinity of antibodies is by site directed mutagenesis. The majority of antigen contacting amino acid side chains of antibodies are usually located in the complementarity determining regions (CDRs), the V_{H} (CDR1, CDR2, and CDR3) and the V_{L} (CDR1, CDR2, and CDR3) (Chothia et al. (1987) J. Mol. Biol., 196: 901-917; Chothia et al. (1986) Science, 233: 755-8; Nhan et al. (1991) J. Mol. Biol., 217: 133-151). These residues contribute the majority of binding energetics responsible for antibody affinity for antigen. In other molecules, mutating amino acids that contact ligand has been shown to be an effective means of increasing the affinity of one protein molecule for its binding partner (Lowman et al. (1993) J Mol. Biol., 234: 564-578; Wells (1990) Biochemistry, 29: 8509-8516). Thus mutation (randomization) of the CDRs and screening against GREB1, or the epitopes thereof identified herein, may be used to generate GREB1 antibodies having improved binding affinity.

In a preferred embodiment, each CDR is randomized in a separate library, using, for example, a GREB1 antibody as a template. To simplify affinity measurement, lower affinity GREB1 antibodies, are used as a template, rather than a higher affinity scFv. The CDR sequences of the highest affinity mutants from each CDR library are combined to obtain an additive increase in affinity. A similar approach has been used to increase the affinity of human growth hormone (hGH) for the growth hormone receptor over 1500 fold from 3.4×10⁻¹⁰ to 9.0×10⁻¹³ M (Lowman et al. (1993) J. Mol. Biol., 234: 564-578).

To increase the affinity of GREB1 antibodies, amino acid residues located in one or more CDRs are partially randomized by synthesizing a ‘doped’ oligonucleotide in which the wild type nucleotide occurs with certain identifiable frequency, e.g. 50%. The oligonucleotide would then be used to amplify the remainder of the GREB1 scFv gene(s) using PCR.

To select higher affinity mutant scFv, each round of selection of the phage antibody libraries is conducted on decreasing amounts of GREB1 antibody.

In another preferred embodiment, GREB1 homodimers are provided. For example, to create GREB1 (scFv′)₂ antibodies, two GREB1 scFvs are joined, either through a linker (e.g., a carbon linker, a peptide, etc.) or through a disulfide bond between, for example, two cysteines. Thus, for example, to create disulfide linked GREB1 scFv, a cysteine residue can be introduced by site directed mutagenesis between the myc tag and hexahistidine tag at the carboxy-terminus of the GREB1 scFv. Introduction of the correct sequence is verified by DNA sequencing. Expressed scFv would have, for example, the myc tag at the C-terminus, followed by glycines, a cysteine, and then 6 histidines to facilitate purification by IMAC. To produce (scFv′)₂ dimers, the cysteine is reduced by incubation with 1 mM beta-mercaptoethanol, and half of the scFv blocked by the addition of DTNB. Blocked and unblocked scFvs are incubated together to form (scFv′)₂ and the resulting material can optionally be analyzed by gel filtration. The affinity of the GREB1 scFv′ monomer and (scFv′)₂ dimer can optionally be determined by BIAcore.

In a preferred embodiment, the (scFv′)₂ dimer is created by joining the scFv fragments through a linker, more preferably through a peptide linker. This can be accomplished by a wide variety of means well known to those of skill in the art. For example, Holliger et al. (1993) Proc. Natl. Acad. Sci. USA, 90: 6444-6448 (see also WO 94/13804).

Typically, linkers are introduced by PCR cloning. For example, synthetic oligonucleotides encoding the 5 amino acid linker (G₄S) can be used to PCR amplify the GREB1 antibody V_{H} and V_{L} genes which are then spliced together to create the GREB1 diabody gene. The gene is then cloned into an appropriate vector, expressed, and purified according to standard methods well known to those of skill in the art.

In preferred embodiments, selection of GREB1 antibodies (whether produced by phage display, immunization methods, hybridoma technology, etc.) involves screening the resulting antibodies for specific binding to an appropriate antigen. For example, GREB1 polypeptides, peptides, variants, isoforms, mutants, and the like.

Selection can by any of a number of methods well known to those of skill in the art. For example, immunochromatography (e.g., using immunotubes, Maxisorp, Nunc) against GREB1.

Selection for increased avidity involves measuring the affinity of a GREB1 antibody (or a modified GREB1 antibody) for GREB1 (or a GREB1 fragment, or an epitope on GREB1, etc.). Methods of making such measurements are known in the art. For example, the Kd of a GREB1 antibody and the kinetics of binding to GREB1 can be determined in a BIAcore, a biosensor based on surface plasmon resonance. For this technique, antigen is coupled to a derivatized sensor chip capable of detecting changes in mass. When antibody is passed over the sensor chip, antibody binds to the antigen resulting in an increase in mass that is quantifiable. Measurement of the rate of association as a function of antibody concentration can be used to calculate the association rate constant (kₒₙ). After the association phase, buffer is passed over the chip and the rate of dissociation of antibody (k_{off}) determined. The equilibrium constant Kd is then calculated as k_{off}/kₒₙ. Affinities measured in this manner correlate well with affinities measured in solution by fluorescence quench titration.

Human or Humanized Antibody Production: As indicated above, the GREB1 antibodies of this invention can be administered to an organism (e.g., a human patient) for various purposes. Antibodies administered to an organism other than the species in which they are raised can be immunogenic. Thus, for example, murine antibodies repeatedly administered to a human often induce an immunologic response against the antibody (e.g., the human anti-mouse antibody (HAMA) response). While this is typically not a problem for the use of non-human antibodies of this invention as they are typically not utilized repeatedly, the immunogenic properties of the antibody are reduced by altering portions, or all, of the antibody into characteristically human sequences thereby producing chimeric or human antibodies, respectively.

Humanized antibodies are immunoglobulin molecules comprising a human and non-human portion. More specifically, the antigen combining region (or variable region) of a humanized chimeric antibody is derived from a non-human source (e.g., murine) and the constant region of the chimeric antibody (which confers biological effector function to the immunoglobulin) is derived from a human source. The humanized chimeric antibody should have the antigen binding specificity of the non-human antibody molecule and the effector function conferred by the human antibody molecule. A large number of methods of generating chimeric antibodies are well known to those of skill in the art (see, e.g., U.S. Pat. Nos. 5,502,167, 5,500,362, 5,491,088, 5,482,856, 5,472,693, 5,354,847, 5,292,867, 5,231,026, 5,204,244, 5,202,238, 5,169,939, 5,081,235, 5,075,431, and 4,975,369).

In general, the procedures used to produce humanized antibodies consist of the following steps (the order of some steps may be interchanged): (a) identifying and cloning the correct gene segment encoding the antigen binding portion of the antibody molecule; this gene segment (known as the VDJ, variable, diversity and joining regions for heavy chains or VJ, variable, joining regions for light chains (or simply as the V or variable region) may be in either the cDNA or genomic form; (b) cloning the gene segments encoding the constant region or desired part thereof; (c) ligating the variable region to the constant region so that the complete chimeric antibody is encoded in a transcribable and translatable form; (d) ligating this construct into a vector containing a selectable marker and gene control regions such as promoters, enhancers and poly(A) addition signals; (e) amplifying this construct in a host cell (e.g., bacteria); (f) introducing the DNA into eukaryotic cells (transfection) most often mammalian lymphocytes; and culturing the host cell under conditions suitable for expression of the chimeric antibody.

In one embodiment, a recombinant DNA vector is used to transfect a cell line that produces a GREB1 antibody. The novel recombinant DNA vector contains a “replacement gene” to replace all or a portion of the gene encoding the immunoglobulin constant region in the cell line (e.g., a replacement gene may encode all or a portion of a constant region of a human immunoglobulin, a specific immunoglobulin class, or an enzyme, a toxin, a biologically active peptide, a growth factor, inhibitor, or a linker peptide to facilitate conjugation to a drug, toxin, or other molecule, etc.), and a “target sequence” which allows for targeted homologous recombination with immunoglobulin sequences within the antibody producing cell.

In another embodiment, a recombinant DNA vector is used to transfect a cell line that produces an antibody having a desired effector function, (e.g., a constant region of a human immunoglobulin) in which case, the replacement gene contained in the recombinant vector may encode all or a portion of a region of an GREB1 antibody and the target sequence contained in the recombinant vector allows for homologous recombination and targeted gene modification within the antibody producing cell. In either embodiment, when only a portion of the variable or constant region is replaced, the resulting chimeric antibody may define the same antigen and/or have the same effector function yet be altered or improved so that the humanized antibody may demonstrate a greater antigen specificity, greater affinity binding constant, increased effector function, or increased secretion and production by the transfected antibody producing cell line, etc. Regardless of the embodiment practiced, the processes of selection for integrated DNA (via a selectable marker), screening for humanized antibody production, and cell cloning, can be used to obtain a clone of cells producing the humanized antibody.

Thus, a piece of DNA which encodes a modification for a monoclonal antibody can be targeted directly to the site of the expressed immunoglobulin gene within a B-cell or hybridoma cell line. DNA constructs for any particular modification may be used to alter the protein product of any monoclonal cell line or hybridoma. Such a procedure circumvents the costly and time consuming task of cloning both heavy and light chain variable region genes from each B-cell clone expressing a useful antigen specificity. In addition to circumventing the process of cloning variable region genes, the level of expression of humanized antibody should be higher when the gene is at its natural chromosomal location rather than at a random position. Detailed methods for preparation of humanized antibodies can be found in U.S. Pat. No. 5,482,856.

Human Antibodies: In another embodiment, this invention provides for fully human anti-GREB1 antibodies. Human antibodies consist entirely of characteristically human polypeptide sequences. The human GREB1 antibodies of this invention can be produced in using a wide variety of methods (see, e.g., Larrick et al., U.S. Pat. No. 5,001,065, for review).

In one preferred embodiment, fully human antibodies are produced using phage display methods. However, instead of utilizing a murine gene library, a human gene library is used. Methods of producing fully human gene libraries are well known to those of skill in the art (see, e.g., Vaughn et al. (1996) Nature Biotechnology, 14(3): 309-314, Marks et al. (1991) J. Mol. Biol., 222: 581-597). For example, the human GREB1 antibodies are initially introduced to trioma cells. Genes encoding the antibodies are then cloned and expressed in other cells, particularly, nonhuman mammalian cells. The general approach for producing human antibodies by trioma technology was originally described by Ostberg et al. (1983) Hybridoma 2: 361-367, Ostberg, U.S. Pat. No. 4,634,664, and Engelman et al., U.S. Pat. No. 4,634,666. The antibody-producing cell lines obtained by this method are called triomas because they are descended from three cells; two human and one mouse. Triomas have been found to produce antibody more stably than ordinary hybridomas made from human cells. Preparation of trioma cells requires an initial fusion of a mouse myeloma cell line with unimmunized human peripheral B lymphocytes. This fusion generates a xenogenic hybrid cell containing both human and mouse chromosomes (see, Engelman, supra). Xenogenic cells that have lost the capacity to secrete antibodies are selected. Preferably, a xenogenic cell is selected that is resistant to 8-azaguanine Such cells are unable to propagate on hypoxanthine-aminopterin-thymidine (HAT) or azaserine-hypoxanthine (AH) media.

The capacity to secrete antibodies is conferred by a further fusion between the xenogenic cell and B-lymphocytes immunized against an GREB1 polypeptide (e.g., GREB1, or GREB1 subsequences including, but not limited to subsequences comprising GREB1 mutants, variants, isoforms etc). The B-lymphocytes are obtained from the spleen, blood or lymph nodes of human donor. If antibodies against a specific antigen or epitope are desired, it is preferable to use that antigen or epitope thereof as the immunogen rather than the entire polypeptide. Alternatively, B-lymphocytes are obtained from an unimmunized individual and stimulated with a GREB1 polypeptide, or an epitope thereof, in vitro. In a further variation, B-lymphocytes are obtained from an infected, or otherwise immunized individual, and then hyperimmunized by exposure to a GREB1 polypeptide for about seven to fourteen days, in vitro.

The immunized B-lymphocytes prepared by one of the above procedures are fused with a xenogenic hybrid cell by well known methods. For example, the cells are treated with 40-50% polyethylene glycol of MW 1000-4000, at about 37° C. for about 5-10 min. Cells are separated from the fusion mixture and propagated in media selective for the desired hybrids. When the xenogenic hybrid cell is resistant to 8-azaguanine, immortalized trioma cells are conveniently selected by successive passage of cells on HAT or AH medium. Other selective procedures are, of course, possible depending on the nature of the cells used in fusion. Clones secreting antibodies having the required binding specificity are identified by assaying the trioma culture medium for the ability to bind to the GREB1 polypeptide or an epitope thereof. Triomas producing human antibodies having the desired specificity are subcloned by the limiting dilution technique and grown in vitro in culture medium, or are injected into selected host animals and grown in vivo. The trioma cell lines obtained are then tested for the ability to bind a GREB1 polypeptide or an epitope thereof. Antibodies are separated from the resulting culture medium or body fluids by conventional antibody-fractionation procedures, such as ammonium sulfate precipitation, DEAE cellulose chromatography and affinity chromatography.

Although triomas are genetically stable they do not produce antibodies at very high levels. Expression levels can be increased by cloning antibody genes from the trioma into one or more expression vectors, and transforming the vector into a cell line such as the cell lines typically used for expression of recombinant or humanized immunoglobulins. As well as increasing yield of antibody, this strategy offers the additional advantage that immunoglobulins are obtained from a cell line that does not have a human component, and does not therefore need to be subjected to the especially extensive viral screening required for human cell lines.

The genes encoding the heavy and light chains of immunoglobulins secreted by trioma cell lines are cloned according to methods, including but not limited to, the polymerase chain reaction (PCR), known in the art (see, e.g., Sambrook et al., Molecular Cloning: A Laboratory Manual, 2nd ed., Cold Spring Harbor, N.Y., 1989; Berger & Kimmel, Methods in Enzymology, Vol. 152: Guide to Molecular Cloning Techniques, Academic Press, Inc., San Diego, Calif., 1987; Co et al. (1992) J Immunol., 148: 1149).

Typically, recombinant constructs comprise DNA segments encoding a complete human immunoglobulin heavy chain and/or a complete human immunoglobulin light chain of an immunoglobulin expressed by a trioma cell line. Alternatively, DNA segments encoding only a portion of the primary antibody genes are produced, which portions possess binding and/or effector activities. Other recombinant constructs contain segments of trioma cell line immunoglobulin genes fused to segments of other immunoglobulin genes, particularly segments of other human constant region sequences (heavy and/or light chain). Human constant region sequences can be selected from various reference sources, including but not limited to those listed in Kabat et al. Sequences of Proteins of Immunological Interest, U.S. Department of Health and Human Services.

In addition to the DNA segments encoding GREB1 immunoglobulins or fragments thereof, other substantially homologous modified immunoglobulins can be readily designed and manufactured utilizing various recombinant DNA techniques known to those skilled in the art such as site-directed mutagenesis (see Gillman & Smith (1979) Gene, 8: 81-97; Roberts et al. (1987) Nature 328: 731-734). Such modified segments will usually retain antigen binding capacity and/or effector function. Moreover, the modified segments are usually not so far changed from the original trioma genomic sequences to prevent hybridization to these sequences under stringent conditions. Because, like many genes, immunoglobulin genes contain separate functional regions, each having one or more distinct biological activities, the genes may be fused to functional regions from other genes to produce fusion proteins (e.g., immunotoxins) having novel properties or novel combinations of properties. The genomic sequences can be cloned and expressed according to standard methods as described herein.

Aptamers: GREB1 specific molecules can be in the form of aptamers. “Aptamers” are DNA or RNA molecules that have been selected from random pools based on their ability to bind other molecules. The aptamer binds specifically to a target molecule wherein the nucleic acid molecule has sequence that comprises a sequence recognized by the target molecule in its natural setting. Alternately, an aptamer can be a nucleic acid molecule that binds to a target molecule wherein the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art (see, e.g., Gold et al., Annu. Rev. Biochem. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chem. 45:1628, 1999).

As used herein, the term “aptamer” or “selected nucleic acid binding species” shall include non-modified or chemically modified RNA or DNA. The method of selection may be by, but is not limited to, affinity chromatography and the method of amplification by reverse transcription (RT) or polymerase chain reaction (PCR).

### Diagnostics and Therapies

Antibodies to GREB1 can be used in established procedures, e.g., to detect breast cancer cells in situ, in biopsies, or in immunohistological procedures.

Preferably, an antibody to GREB1 is used in a qualitative (GREB1 present or absent) or quantitative (GREB1 amount is determined) immunoassay.

In preferred embodiments, the expression and/or activity of GREB1 in vivo is monitored and correlated with disease progression and/or prognosis of disease outcome.

In another preferred embodiment, the detection of GREB1 and/or the expression profile of GREB1 is correlated with identifying individuals or subjects at risk of developing a disease or disorder associated with GREB1 expression, expression profiles and/or activity as compared to normal controls.

Measuring the level of protein GREB1 is very advantageous in the detection of cancer, such as for example, breast cancer, prostrate cancer. Therefore, in a further preferred embodiment, the present invention relates to use of protein GREB1 and/or GREB1 antibodies or apatmers as a marker molecule in the diagnosis of cancer from a biological sample obtained from an individual.

In a preferred embodiment, it is especially preferred to use the novel marker GREB1 in the early diagnosis of breast cancer.

In another preferred embodiment, it is especially preferred to use the novel marker GREB1 in the early diagnosis of prostrate cancer.

The GREB1 biomarkers can be correlated with any quantifiable signs, symptoms and/or analytes in biological samples characteristic of a particular disease, in this case cancer. The diagnostic and prognostic methods relate to the identification and evaluation of the biomarkers, both individually and, optionally, as they relate to other biomarkers. For example, the biomarker expression, function, activity, etc. in a patient can be correlated with all types of biological data from a patient in the prognosis, diagnosis, outcome of a disease, identification of individuals at risk of developing a disease, such as cancer.

The patient data may include a variety of types of data which have some association with the disease. The information may be biological. Such data may be derived from measurement of any biological parameter. Such substances include, but are not limited to, endocrine substances such as hormones, exocrine substances such as enzymes, and neurotransmitters, electrolytes, proteins, carbohydrates, growth factors, cytokines, monokines, fatty acids, triglycerides, and cholesterol.

Other types of biological data may be derived from histological analysis of organs, tissues or cells removed from patients, including histological analyses performed at the light microscopic and electron microscopic levels utilizing any number of techniques including, but not limited to, structural analysis, histochemical, immunocytochemical, in situ hybridization, and autoradiographic techniques.

Biological data may be derived from analysis of cells removed from patients and grown in culture. Various characteristics of these cells may be examined histologically and biochemically. For example, cells removed from a patient and placed in culture may be examined for the presence of specific markers associated with the presence of a disease. Cells may be examined for their metabolic activity or for the products made and released into the culture medium.

Biological data about a patient includes results from genetic and molecular biological analysis of the nuclear and cytoplasmic molecules associated with transcription and translation such as various forms of ribonucleic acid, deoxyribonucleic acid and other transcription factors, and the end product molecules resulting from the translation of such ribonucleic acid molecules.

Also included in the category of biological data are the various structural and anatomical analytical methods used with patients such as radiographs, mammograms, fluorographs and tomographs, including but not limited to X-ray, magnetic resonance imaging, computerized assisted tomography, visualization of radiopaque materials introduced into the body, positron emission tomography, endoscopy, sonograms, echocardiograms, and improvements thereof.

Biological data also includes data concerning the age, height, growth rate, dental health, cardiovascular status, reproductive status (pre-pubertal, pubertal, post-pubertal, pre-menopausal, menopausal, post-menopausal, fertile, infertile), body fat percentage, and body fat distribution. Biological data also includes the results of physical examinations, including but not limited to manual palpation, digital rectal examination, prostate palpation, testicular palpation, weight, body fat amount and distribution, auscultation, testing of reflexes, blood pressure measurements, heart and related cardiovascular sounds, prostrate and testicular examinations, vaginal and other gynecologic examinations, including cervical, uterine and ovarian palpation, evaluation of the uterine tubes, breast examinations, and radiographic and infrared examination of the breasts.

Additional biological data can be obtained in the form of a medical history of the patient. Such data includes, but is not limited to the following: medical history of ancestors including grandparents and parents, siblings, and descendants, their medical problems, genetic histories, psychological profiles, psychiatric disease, age at death and cause of death; prior diseases and conditions; prior surgeries; prior angioplasties, vaccinations; habits such as exercise schedules, alcohol consumption, cigarette consumption and drug consumption; cardiac information including but not limited to blood pressure, pulse, electrocardiogram, echocardiogram, coronary arteriogram, treadmill stress tests, thallium stress tests and other cardiovascular imaging techniques. All of the aforementioned types of biological data can be considered in conjunction with the detection or absence of the GREB1 biomarkers.

The use of protein GREB1 itself, represents a significant progress to the challenging field of cancer diagnosis. Combining measurements of GREB1 with other known markers, e.g. CA 15-3, CEA, cellular retinoic acid-binding protein or with other markers of presently known or yet to be discovered, leads to further improvements. Therefore, in a further preferred embodiment the present invention relates to the use of GREB1 as a marker molecule for cancer in combination with one or more marker molecules for cancer in the diagnosis of cancer from a biological sample obtained from an individual. In this regard, the expression “one or more” denotes 1 to 20, preferably 1 to 15, preferably 1 to 10.

Preferred selected other markers with which the measurement of GREB1 may be combined are tumor antigens comprising: HER2, HER3, Muc-1, EGFR, PSMA, CD20, CD22, CD23, TAA, GDR antigens, VEGFR and the like.

Other non-limiting examples of tumor antigens, include, tumor antigens resulting from mutations, such as: alpha-actinin-4 (lung carcinoma); CASP-8 (head and neck squamous cell carcinoma); beta-catenin (melanoma); Cdc27 (melanoma); CDK4 (melanoma); Elongation factor 2 (lung squamous carcinoma); LDLR-fucosyltransferaseAS fusion protein (melanoma); overexpression of HLA-A2^{d} (renal cell carcinoma); hsp70-2 (renal cell carcinoma); KIAAO205 (bladder tumor); MART2 (melanoma); MUM-1f (melanoma); MUM-2 (melanoma); MUM-3 (melanoma); neo-PAP (melanoma); Myosin class I (melanoma); OS-9g (melanoma); PTPRK (melanoma). Examples of differentiation tumor antigens include, but not limited to: CEA (gut carcinoma); gp100/Pmel17 (melanoma); Kallikrein 4 (prostate); mammaglobin-A (breast cancer); Melan-A/MART-1 (melanoma); PSA (prostate carcinoma); TRP-1/gp75 (melanoma); TRP-2 (melanoma); tyrosinase (melanoma). Over or under-expressed tumor antigens include but are not limited to: CPSF (ubiquitous); EphA3; G250/MN/CAIX (stomach, liver, pancreas); HER-2/neu; Intestinal carboxyl esterase (liver, intestine, kidney); alpha-fetoprotein (liver); M-CSF (liver, kidney); MUC1 (glandular epithelia); p53 (ubiquitous); PRAME (testis, ovary, endometrium, adrenals); PSMA (prostate, CNS, liver); RAGE-1 (retina); RU2AS (testis, kidney, bladder); survivin (ubiquitous); Telomerase (testis, thymus, bone marrow, lymph nodes); WT1 (testis, ovary, bone marrow, spleen); CAl25 (ovarian).

Preferably, the inventive method is used with samples of patients suspected of suffering from breast cancer or prostrate cancer. However, the GREB1 biomarkers can be as a stand alone marker or combined with other markers for diagnosis, prognosis, assesses the possible severity of disease progression, or assesses risk of an individual developing cancer comprising: cancers of the colon, lung, stomach, ovary, pancreatic, liver, kidney, brain and the like.

In accordance with one embodiment of the present invention, a biological sample or several biological samples are first collected from a patient. The GREB1 biomarkers and/or other biomarkers associated with a specific disease are measured in the biological samples using standard laboratory techniques, to determine their concentrations, or in some cases their presence or absence. It is to be understood that this process can be carried out automatically in conventional diagnostic machines.

In a preferred embodiment, a method for the diagnosis of cancer comprises the steps of a) providing a biological sample obtained from an individual suspected of suffering from cancer, b) contacting said sample with a specific binding agent for GREB1 under conditions appropriate for formation of a complex between said binding agent and GREB1, and/or expression or lack thereof of GREB1 molecules c) correlating the amount of complex formed and/or expression levels of GREB1 in (b) to the diagnosis of cancer. The diagnosis includes accurate diagnosis and prognosis of cancer, even at very early stages of the disease.

In another preferred embodiment, a method of predicting the outcome or severity of a cancer comprises a) providing a biological sample obtained from an individual suspected of suffering from cancer, b) contacting said sample with a specific binding agent for GREB1 under conditions appropriate for formation of a complex between said binding agent and GREB1, and/or expression or lack thereof of GREB1 molecules c) correlating the amount of complex formed and/or expression levels of GREB1 in (b) to the predicting the outcome or severity of a cancer. The samples can be taken over periods of time and compared to normal controls.

In some embodiments, the expression profiles over time or variations in the expression profiles depending on the in vivo source of the GREB1 expression profiles can be used as a predictor of disease, such as breast or prostrate cancer.

In another preferred embodiment, identification of individuals at risk of developing cancer comprises a) providing a biological sample obtained from an individual suspected of suffering from cancer, b) contacting said sample with a specific binding agent for GREB1 under conditions appropriate for formation of a complex between said binding agent and GREB1, and/or expression or lack thereof of GREB1 molecules c) correlating the amount of complex formed and/or expression levels of GREB1 in (b) to the predicting the outcome or severity of a cancer. The samples can be taken over periods of time and compared to normal controls.

Diagnostic reagents in the field of specific binding assays, like immunoassays, usually are best provided in the form of a kit, which comprises the specific binding agent and the auxiliary reagents required to perform the assay. The present invention therefore also relates to an immunological kit comprising at least one specific binding agent for GREB1 and auxiliary reagents for measurement of GREB1. Also preferred is an immunological kit comprising at least one specific binding agent for GREB1, at least one specific binding agent for another marker, e.g. CA 15-3 and auxiliary reagents for measurement of GREB1 and CA 15-3.

Accuracy of a test is best described by its receiver-operating characteristics (ROC) (see especially Zweig, M. H., and Campbell, G., Clin. Chem. 39 (1993) 561-577). The ROC graph is a plot of all of the sensitivity/specificity pairs resulting from continuously varying the decision thresh-hold over the entire range of data observed. The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease or benign versus malignant disease.

Therapies: In a preferred embodiment, a method of treating a patient suffering from or at risk of developing cancer comprises administration of an effective amount of a drug or molecule which kills the cancer cells. The effect of the treatment can be monitored, inter alia, by determining the expression and/or function of GREB1 markers and/or detection of GREB1 antibodies.

In one preferred embodiment, the GREB1 antibody or binding fragment thereof, is used as a targeting domain to transport a therapeutic effector domain. For example, the antibody can be a fragment which specifically binds to GREB1 epitopes and is conjugated to or is a fusion protein in which one of the domains comprises a therapeutic effector domain.

Examples of such domains include, cytolytic molecules that can be used to fuse to the antibody or fragment thereof, such as for example, TNF-α, TNF-β, peptide toxins—such as ricin, abrin, diphtheria, gelonin, Pseudomonas exotoxin A, Crotalus durissus terrificus toxin, Crotalus adamenteus toxin, Naja naja toxin, and Naja mocambique toxin. (Hughes et al., Hum. Exp. Toxicol. 15:443, 1996; Rosenblum et al., Cancer Immunol. Immunother. 42:115, 1996; Rodriguez et al., Prostate 34:259, 1998; Mauceri et al., Cancer Res. 56:4311; 1996).

Also suitable are molecules that induce or mediate apoptosis—such as the ICE-family of cysteine proteases, the Bcl-2 family of proteins, Bax, BclXs and caspases (Favrot et al., Gene Ther. 5:728, 1998; McGill et al., Front. Biosci. 2:D353, 1997; McDonnell et al., Semin. Cancer Biol. 6:53, 1995). Another potential anti-tumor agent is apoptin, a protein that induces apoptosis even where small drug chemotherapeutics fail (Pietersen et al., Adv. Exp. Med. Biol. 465:153, 2000). Koga et al. (Hu. Gene Ther. 11: 1397, 2000) propose a telomerase-specific gene therapy using the hTERT gene promoter linked to the apoptosis gene Caspase-8 (FLICE).

Also of interest are enzymes present in the lytic package that cytotoxic T lymphocytes or LAK cells deliver to their targets. Perforin, a pore-forming protein, and Fas ligand are major cytolytic molecules in these cells (Brandau et al., Clin. Cancer Res. 6:3729, 2000; Cruz et al., Br. J. Cancer 81:881, 1999). CTLs also express a family of at least 11 serine proteases termed granzymes, which have four primary substrate specificities (Kam et al., Biochim. Biophys. Acta 1477:307, 2000). Low concentrations of streptolysin 0 and pneumolysin facilitate granzyme B-dependent apoptosis (Browne et al., Mol. Cell Biol. 19:8604, 1999).

Other suitable effectors encode polypeptides having activity that is not itself toxic to a cell, but renders the cell sensitive to an otherwise nontoxic compound—either by metabolically altering the cell, or by changing a non-toxic prodrug into a lethal drug. Exemplary is thymidine kinase (tk), such as may be derived from a herpes simplex virus, and catalytically equivalent variants. The HSV tk converts the anti-herpetic agent ganciclovir (GCV) to a toxic product that interferes with DNA replication in proliferating cells.

Other domains include, but not limited to, chemokines, cytokines, e.g. interleukins, e.g. IL-2, IL-3, IL-6, and IL-11, as well as the other interleukins, the colony stimulating factors, such as GM-CSF, interferons, e.g. γ-interferon, erythropoietin.

In connection solid tumor treatment, the present invention may be used in combination with classical approaches, such as surgery, radiotherapy, chemotherapy, and the like. The invention therefore provides combined therapies in which the therapeutic compositions are used simultaneously with, before, or after surgery or radiation treatment; or are administered to patients with, before, or after conventional chemotherapeutic, radiotherapeutic or other anti-angiogenic agents, or targeted immunotoxins or coaguligands.

In some embodiments the method according to this aspect of the invention further involves administering to the subject an anti-tumor medicament. As used herein, an “anti-tumor medicament” or, equivalently, a “cancer medicament”, refers to an agent which is administered to a subject for the purpose of treating a cancer. As used herein, “treating cancer” includes preventing the development of a cancer, reducing the symptoms of cancer, and/or inhibiting the growth of an established cancer. In other aspects, the cancer medicament is administered to a subject at risk of developing a cancer for the purpose of reducing the risk of developing the cancer. Various types of medicaments for the treatment of cancer are described herein. For the purpose of this specification, cancer medicaments are classified as chemotherapeutic agents, immunotherapeutic agents, cancer vaccines, hormone therapy, and biological response modifiers. Additionally, the methods of the invention are intended to embrace the use of more than one cancer medicament along with the GREB1-binding molecule of the present invention. As an example, where appropriate, the GREB1-binding molecule can be administered with a both a chemotherapeutic agent and an immunotherapeutic agent. Alternatively, the cancer medicament can embrace an immunotherapeutic agent and a cancer vaccine, or a chemotherapeutic agent and a cancer vaccine, or a chemotherapeutic agent, an immunotherapeutic agent and a cancer vaccine all administered to one subject for the purpose of treating a subject having a cancer or at risk of developing a cancer.

Cancer medicaments function in a variety of ways. Some cancer medicaments work by targeting physiological mechanisms that are specific to tumor cells. Cancer medicaments can target signal transduction pathways and molecular mechanisms which are altered in cancer cells. Targeting of cancer cells via the epitopes expressed on their cell surface is accomplished through the use of monoclonal antibodies such as for example GREB1 antibodies.

As used herein, chemotherapeutic agents embrace all other forms of cancer medicaments which do not fall into the categories of immunotherapeutic agents or cancer vaccines. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation.

Chemotherapeutic agents which are currently in development or in use in a clinical setting include, without limitation: 5-FU Enhancer, 9-AC, AG2037, AG3340, Aggrecanase Inhibitor, Aminoglutethimide, Amsacrine (m-AMSA), Angiogenesis Inhibitor, Anti-VEGF, Asparaginase, Azacitidine, Batimastat (BB94), BAY 12-9566, BCH-4556, Bis-Naphtalimide, Busulfan, Capecitabine, Carboplatin, Carmustaine+Polifepr Osan, cdk4/cdk2 inhibitors, Chlorombucil, CI-994, Cisplatin, Cladribine, CS-682, Cytarabine HCl, D2163, Dactinomycin, Daunorubicin HCl, DepoCyt, Dexifosamide, Docetaxel, Dolastain, Doxifluridine, Doxorubicin, DX8951f, E 7070, EGFR, Epirubicin, Erythropoietin, Estramustine phosphate sodium, Etoposide (VP16-213), Farnesyl Transferase Inhibitor, FK 317, Flavopiridol, Floxuridine, Fludarabine, Fluorouracil (5-FU), Flutamide, Fragyline, Gemcitabine, Hexamethylmelamine (HMM), Hydroxyurea (hydroxycarbamide), Ifosfamide, Interferon Alfa-2a, Interferon Alfa-2b, Interleukin-2, Irinotecan, ISI 641, Krestin, Lemonal DP 2202, Leuprolide acetate (LHRH-releasing factor analogue), Levamisole, LiGLA (lithium-gamma linolenate), Lodine Seeds, Lometexol, Lomustine (CCNU), Marimistat, Mechlorethamine HCl (nitrogen mustard), Megestrol acetate, Meglamine GLA, Mercaptopurine, Mesna, Mitoguazone (methyl-GAG; methyl glyoxal bis-guanylhydrazone; MGBG), Mitotane (o.p-DDD), Mitoxantrone, Mitoxantrone HCl, MMI 270, MMP, MTA/LY 231514, Octreotide, ODN 698, OK-432, Oral Platinum, Oral Taxoid, Paclitaxel (TAXOL™), PARP Inhibitors, PD 183805, Pentostatin (2′ deoxycoformycin), PKC 412, Plicamycin, Procarbazine HCl, PSC 833, Ralitrexed, RAS Farnesyl Transferase Inhibitor, RAS Oncogene Inhibitor, Semustine (methyl-CCNU), Streptozocin, Suramin, Tamoxifen citrate, Taxane Analog, Temozolomide, Teniposide (VM-26), Thioguanine, Thiotepa, Topotecan, Tyrosine Kinase, UFT (Tegafur/luracil), Valrubicin, VEGF/b-FGF Inhibitors, Vinblastine sulfate, Vindesine sulfate, VX-710, VX-853, YM 116, ZD 0101, ZD 0473/Anormed, ZD 1839, ZD 9331.

### Candidate Agents

In another preferred embodiment, the invention provides assays, preferably high-throughput screening assays for the identification of candidate therapeutic agents in the treatment of diseases associated with abnormal GREB1 expression and/or function. In one embodiment, a disease associated with abnormal GREB1 expression and/or function is cancer, such as breast or prostrate cancer.

In another preferred embodiment, methods (also referred to herein as “screening assays”) are provided for identifying modulators, i.e., candidate or test compounds or agents (e.g., proteins, peptides, peptidomimetics, peptoids, small molecules or other drugs) which modulate the expression, function, activity of GREB1. Compounds thus identified can be used to modulate the activity of target gene products, e.g. GREB1 gene products, prolong the half-life of a protein or peptide, regulate cell division, etc, in a therapeutic protocol, to elaborate the biological function of the target gene product, or to identify compounds that disrupt normal target gene interactions.

An agent identified by the methods of the invention may be a small molecule, a chemical, a peptide, a peptidomimetic, organic or inorganic molecules.

After identifying a test compound or candidate agent as an agonist and/or an antagonist, the compound may then be used to treat subjects with diseases and disorders associated with GREB1 or hormonal activity.

Candidate agents include numerous chemical classes, though typically they are organic compounds including small organic compounds, nucleic acids including oligonucleotides, and peptides. Small organic compounds suitably may have e.g. a molecular weight of more than about 40 or 50 yet less than about 2,500. Candidate agents may comprise functional chemical groups that interact with proteins and/or DNA.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the one-bead one-compound library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Nat'l Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In another preferred embodiment, the candidate therapeutic agent comprises proteins, peptides, organic molecules, inorganic molecules, nucleic acid molecules, and the like. These molecules can be natural, e.g. from plants, fungus, bacteria etc., or can be synthesized or synthetic.

A prototype compound may be believed to have therapeutic activity on the basis of any information available to the artisan. For example, a prototype compound may be believed to have therapeutic activity on the basis of information contained in the Physician's Desk Reference. In addition, by way of non-limiting example, a compound may be believed to have therapeutic activity on the basis of experience of a clinician, structure of the compound, structural activity relationship data, EC₅₀, assay data, IC₅₀ assay data, animal or clinical studies, or any other basis, or combination of such bases.

A therapeutically-active compound is a compound that has therapeutic activity, including for example, the ability of a compound to induce a specified response when administered to a subject or tested in vitro. Therapeutic activity includes treatment of a disease or condition, including both prophylactic and ameliorative treatment. Treatment of a disease or condition can include improvement of a disease or condition by any amount, including prevention, amelioration, and elimination of the disease or condition. Therapeutic activity may be conducted against any disease or condition, including in a preferred embodiment against human immunodeficiency virus, cancer, arthritis or any combination thereof. In order to determine therapeutic activity any method by which therapeutic activity of a compound may be evaluated can be used. For example, both in vivo and in vitro methods can be used, including for example, clinical evaluation, EC₅₀, and IC₅₀ assays, and dose response curves.

Candidate compounds for use with an assay of the present invention or identified by assays of the present invention as useful pharmacological agents can be pharmacological agents already known in the art or variations thereof or can be compounds previously unknown to have any pharmacological activity. The candidate compounds can be naturally occurring or designed in the laboratory. Candidate compounds can comprise a single diastereomer, more than one diastereomer, or a single enantiomer, or more than one enantiomer.

Candidate compounds can be isolated, from microorganisms, animals or plants, for example, and can be produced recombinantly, or synthesized by chemical methods known in the art. If desired, candidate compounds of the present invention can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries. The other four approaches are applicable to polypeptide, non-peptide oligomer, or small molecule libraries of compounds and are preferred approaches in the present invention. See Lam, Anticancer Drug Des. 12: 145-167 (1997).

In an embodiment, the present invention provides a method of identifying a candidate compound as a suitable prodrug. A suitable prodrug includes any prodrug that may be identified by the methods of the present invention. Any method apparent to the artisan may be used to identify a candidate compound as a suitable prodrug.

In another aspect, the present invention provides methods of screening candidate compounds for suitability as therapeutic agents. Screening for suitability of therapeutic agents may include assessment of one, some or many criteria relating to the compound that may affect the ability of the compound as a therapeutic agent. Factors such as, for example, efficacy, safety, efficiency, retention, localization, tissue selectivity, degradation, or intracellular persistence may be considered. In an embodiment, a method of screening candidate compounds for suitability as therapeutic agents is provided, where the method comprises providing a candidate compound identified as a suitable prodrug, determining the therapeutic activity of the candidate compound, and determining the intracellular persistence of the candidate compound. Intracellular persistence can be measured by any technique apparent to the skilled artisan, such as for example by radioactive tracer, heavy isotope labeling, or LCMS.

In screening compounds for suitability as therapeutic agents, intracellular persistence of the candidate compound is evaluated. In a preferred embodiment, the agents are evaluated for their ability to modulate the protein or peptide intracellular persistence may comprise, for example, evaluation of intracellular residence time or half-life in response to a candidate therapeutic agent. In a preferred embodiment, the half-life of a protein or peptide in the presence or absence of the candidate therapeutic compound in human tissue is determined. Half-life may be determined in any tissue. Any technique known to the art worker for determining intracellular persistence may be used in the present invention. By way of non-limiting example, persistence of a compound may be measured by retention of a radiolabeled or dye labeled substance.

A further aspect of the present invention relates to methods of inhibiting the activity of a condition or disease comprising the step of treating a sample or subject believed to have a disease or condition with a prodrug identified by a compound of the invention. Compositions of the invention act as identifiers for prodrugs that have therapeutic activity against a disease or condition. In a preferred aspect, compositions of the invention act as identifiers for drugs that show therapeutic activity against conditions including for example cancer.

In one embodiment, a screening assay is a cell-based assay in which a cell expresses a GREB1 protein or peptide, GREB1-detectable marker construct or fusion protein construct, for example, GST, luciferase fusion partners, isoforms or mutants thereof, which is contacted with a test compound, and the ability of the test compound to modulate the expression and/or activity of GREB1. Determining the ability of the test compound to modulate can be accomplished by monitoring, for example, immunoassays, blots, pull-down assays, etc, assays described in detail in the Examples section which follows. The cell, for example, can be of mammalian origin, e.g., human.

In another preferred embodiment, the screening assay is a high-throughput screening assay.

In another preferred embodiment, soluble and/or membrane-bound forms of isolated proteins, mutants or biologically active portions thereof, can be used in the assays if desired. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, TRITON™ X-100, TRITON™ X-114, THESIT™, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays can also be used and involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al, U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FET binding event can be conveniently measured through standard fluorometric detection means well known in the art (e.g., using a fluorimeter).

In another embodiment, determining the ability of a protein to bind or “dock” to a target molecule or docking site on a target molecule can be accomplished using real-time Biomolecular Interaction Analysis (BIA) (see, e.g., Sjolander, S, and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BLAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target product or the test substance is anchored onto a solid phase. The target product/test compound complexes anchored on the solid phase can be detected at the end of the reaction. Preferably, the target product can be anchored onto a solid surface, and the test compound, (which is not anchored), can be labeled, either directly or indirectly, with detectable labels discussed herein.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Chemical Libraries: Developments in combinatorial chemistry allow the rapid and economical synthesis of hundreds to thousands of discrete compounds. These compounds are typically arrayed in moderate-sized libraries of small molecules designed for efficient screening. Combinatorial methods can be used to generate unbiased libraries suitable for the identification of novel compounds. In addition, smaller, less diverse libraries can be generated that are descended from a single parent compound with a previously determined biological activity. In either case, the lack of efficient screening systems to specifically target therapeutically relevant biological molecules produced by combinational chemistry such as inhibitors of important enzymes hampers the optimal use of these resources.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 90:6909-6913 (1993)); vinylogous polypeptides (Hagihara, et al., J. Amer. Chem. Soc. 114:6568 (1992)); nonpeptidal peptidomimetics with β-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

Small Molecules: Small molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

Data and Analysis: The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention relates to embodiments that include methods for providing genetic information over networks such as the Internet.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention. Embodiments of inventive compositions and methods are illustrated in the following examples.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

### Example 1

### Correlation of GREB1 mRNA with Protein Expression in Breast Cancer: Validation of a Novel GREB1 Monoclonal Antibody

The generation and validation of a novel monoclonal GREB1 antibody (GREB1ab) for applications in immunoblotting as well as immunohistochemical (IHC) methods with normal and diseased breast tissues as well as breast cancer cell lines is provided. This is the first study demonstrating the development and utilization of a monoclonal antibody specifically targeting human GREB1 protein. This antibody will serve, inter alia, as a useful tool for investigations focused on the expression, distribution and function of GREB1 in normal breast and breast cancer tissues.

Materials and Methods

Generation of the GREB1ab: Monoclonal antibodies specific to GREB1 were made in cooperation with ProMab Biotechnologies Inc (www.promab.com). GREB1-His-tagged recombinant protein and GST-tagged truncated-GREB1 protein were synthesized and 2 mg of conjugated His-tagged peptide was used to immunize 5 Balb/c mice. Hybridoma fusion was performed using splenocytes from a mouse with the best titer and SP2/0 myeloma cells. Then supernatants from the growing hybridoma wells were screened by ELISA using the His-tagged GREB1 as an antigen and HRP-labeled anti-IgG (Sigma, Cat#:A0168) as secondary antibody. Ten clones were positive for GREB1ab and subsequently tested by Western blot. Briefly, lysates from the ER⁺, GREB1⁺ MCF-7 breast cancer cell line grown in the presence or absence of estradiol, ICI 182,780, or the combination of estrogen plus ICI 182,780, and the ER⁻, GREB1⁻ MDA-MB-231 cell line were assayed for GREB1 protein expression by Western blot analysis to verify monoclonal antibody specificity and production by the selected clones. Three separate hybridomas were identified by Western blot analysis of lysates from MCF-7 cells grown in estrogen containing media. These three hybridomas were further subcloned by limiting dilution, isotyped, and subsequently expanded to generate sufficient GREB1ab for the experiments described.

Culture of Breast Cancer Cell Lines and Adenoviral Transduction: Breast carcinoma and epithelial cell lines MCF-7, BT-474 and MDA-MB-231 were maintained and growth assays performed as described previously (Rae J M, et al. (2005). Breast Cancer Research and Treatment 92:141-149; Johnson M D, et al. (2004). Breast Cancer Res Treat 85(2):151-159). For defined estrogen culture experiments, cells were washed and grown in steroid depleted media (phenol red-free IMEM supplemented with 5% charcoal stripped calf bovine serum-Valley Biomedical Products, VA) for 3 days and then treated with 1 nM E2 for indicated time points. Recombinant adenovirus containing an empty CMV promoter (Ad-CMV-Null) or expressing GREB1 protein (Ad-GREB1) were obtained from Vector BioLabs (Philadelphia, Pa.). Viral titers were determined by a plaque assay using 293 cells and expressed as numbers of plaque forming units (PFU) per milliliter. Prior to adenovirus infection, MDA-MB-231, MCF-7 cells and 3-day estrogen depleted MCF-7 cells were seeded onto plates, grown to 60% confluence, and infected with Ad-CMV-Null and Ad-GREB1 at an multiplicity of infection (MOI) of 20 viral particles per cell. Twenty-four hours after infection, cells were collected and assayed for GREB1 protein expression.

Expression of siRNA Targeting GREB1 and Controls: Small interfering RNA (siRNA) duplexes (total four pairs) were designed to target human GREB1 mRNA and purchased from Dharmacon (Lafayette, Colo.). A scrambled siRNA with no homology to any known sequence was generated as control. Three-day estrogen-depleted MCF-7 cells were transfected with 100 nM siRNA specific to GREB1 or non-target control using LIPOFECTAMINE™ reagent (Invitrogen, Carlsbad, Calif.) in serum free OptiMEM-1 medium (Invitrogen) according to the manufacture's instructions. After six hours of incubation with transfectants, the cells were split into two groups and grown in 10% CCS for another 24 hours. The cells were subsequently treated with 1 nM E2 or 0.01% ethanol for indicated time periods. All studies were performed in triplicate.

RNA Isolation and Quantitative Real-Time PCR: RNA was extracted from breast cancer cell lines using TRIzol reagent (Invitrogen) according to the manufacturer's protocol. First-strand cDNA was synthesized from total RNA using the SuperScript First-Strand Synthesis System with SuperScript II reverse transcriptase according to the manufacturer's protocols (Invitrogen, Carlsbad, Calif.). The cDNA generated was used as a template in real-time PCR reactions with QuantiTect SYBR-Green PCR master-mix (Bio-RAD) and were run on an ABI PRISM 7700 thermocycler. Real-time quantitative PCR reactions consisted of 1× SybrGreen Supermix (Bio-Rad), 0.25 mmol/L forward and reverse primers, and 10 ng cDNA. Cycling conditions consisted of a three-step amplification and melt curve analysis using the iQ5 Real-time PCR Detection System (Bio-Rad). For generating a standard curve, amplified cDNA from the reference sample detailed above was used in a 5-fold dilution series of 100 to 0.16 ng cDNA per reaction. Relative gene expression was calculated by dividing the specific expression value (starting quantity in ng) by the glyceraldehyde-3-phosphate dehydrogenase expression value. All PCR reactions were repeated in triplicate. Following PCR primers were used: human GREB1 (sense: 5′-CAAAGAATAACCTGTTGGCCCTGC-3′ (SEQ ID NO: 2); antisense: 5′-GACATGCCTGCGCTCTCATACTTA-3′ (SEQ ID NO: 3)); human IRS1 (sense: 5′-CAGAGGACCGTCAGTAGCTCAA-3′ (SEQ ID NO: 4); antisense: 5′-GGAAGATATGAGGTCCTAGTTGTGAAT-3′ (SEQ ID NO: 5)); human IGFBP4 (sense: 5′-AGAGACATGTACCTTGACCATCGTC-3′ (SEQ ID NO: 6); antisense: 5′-GTCTGGACCTCG TGACCATTACT-3′ (SEQ ID NO: 7)); human bcl-2 (sense: 5′-CTCGTCGCTACCGTCGTGACTTCG-3′ (SEQ ID NO: 8); antisense: 5′-CAGATGCCGGTTCAGGTACTCAGTC-3′ (SEQ ID NO: 9)); human glyceraldehyde-3-phosphate dehydrogenase (GAPDH) gene (sense: 5′-GAAGGTGAAGGTCGGAGTC-3′ (SEQ ID NO: 10); antisense: 5′-GAAGATGGTGATGGGATTTC-3′ (SEQ ID NO: 11)).

Western Blotting Analysis of GREB1 Expression: Cells were washed with cold phosphate-buffered saline (PBS) and lysed in 50 mM HEPES (pH 7.5) with 150 mM NaCl-1.5 mM MgCl2-1 mM EGTA-10% glycerol-1% NP-40-100 mM NaF-10 mM sodium pyrophosphate-0.2 mM sodium orthovanadate-1 mM phenylmethylsulfonyl fluoride and 10 μg of aprotinin/ml. After cell lysates were obtained from breast cancer cell lines, aliquots of lysates (100 μg) were resolved by 4-15% SDS-polyacrylamide gels and separated proteins were electrophoretically transferred to nitrocellulose for immunodetection. The membrane was blocked in 5% nonfat dry milk in TBST for one hour at room temperature and incubated with GREB1ab at a dilution of 1:1000 in TBST+5% nonfat dry milk, followed by horseradish peroxidase-conjugated anti-mouse secondary antibody (Amersham) at a dilution of 1:2,000. Immunoblots were re-probed with β-actin monoclonal antibody to confirm equal loading of protein lysates. Western blot assays were conducted in duplicate for each sample.

Immunohistochemical Staining for GREB1, ER and HER2: Breast tumor tissue microarrays (TMA) were provided by Tissue Array Networks (http://Tissue-Array.Net). Grading of the histological malignancy of each specimen was assessed as reported previously (Bloom H J, Richardson W W (1957. British Journal of Cancer 11(3):359-377; Elston C W, Ellis I O (1991) Histopathology 19(5):403-4101. Slides containing formalin-fixed, paraffin-embedded samples were deparaffinized, hydrated in water, and subjected to antigen retrieval in 10 mM citrate buffer, pH 6.0. Immunostaining was performed as described previously with some modifications (Zabel U, et al. (1993) The EMBO Journal 12(1):201-211). Briefly, slides were probed with GREB1ab at a dilution of 1:100 for one hour and subsequently incubated with secondary antibody for another hour. The reaction products were visualized by immersing slides in 3,3-diaminobenzidine tablet sets (Sigma Fast, Sigma) and counterstained with hematoxylin. As negative controls, either the GREB1ab was omitted or sections were incubated with 1×PBS instead of primary antibody. For ER staining, an anti-ER antibody (clone ID5, dilution 1:100; Dako) was employed which is FDA approved. HER2 protein expression status was assessed using an FDA approved IHC assay, the HercepTest (Dako), according to the manufacturer's instructions. TMAs were reviewed and scored by two pathologists blinded to all clinicopathologic data collected on the specimens. GREB1 positivity was defined as >10% stained nuclear area. ER positivity was defined as >10% stained nuclear area (Harvey J M, et al. (1999) J Clin Oncol 17(5):1474-1481). HER2 immunostaining was scored using a 0, 1+, 2+, and 3+ scale with 0, 1+ and 2+ considered low HER2 expression and 3+ delineating over-expression per the American Society of Clinical Oncology/College of American Pathologists guidelines (Wolff AC, et al. J Clin Oncol 25(1):118-145; Wolff AC, et al (2007) Archives of Pathology & Laboratory Medicine 131(1):18-43).

Statistical Analysis: The Statview Software package was used for all calculations. Correlations between categorical variables were performed using the chi-square test and Fisher's exact test. All tests were two-tailed, with a confidence interval of 95%. P values less than 0.05 were considered to be statistically significant.

Results

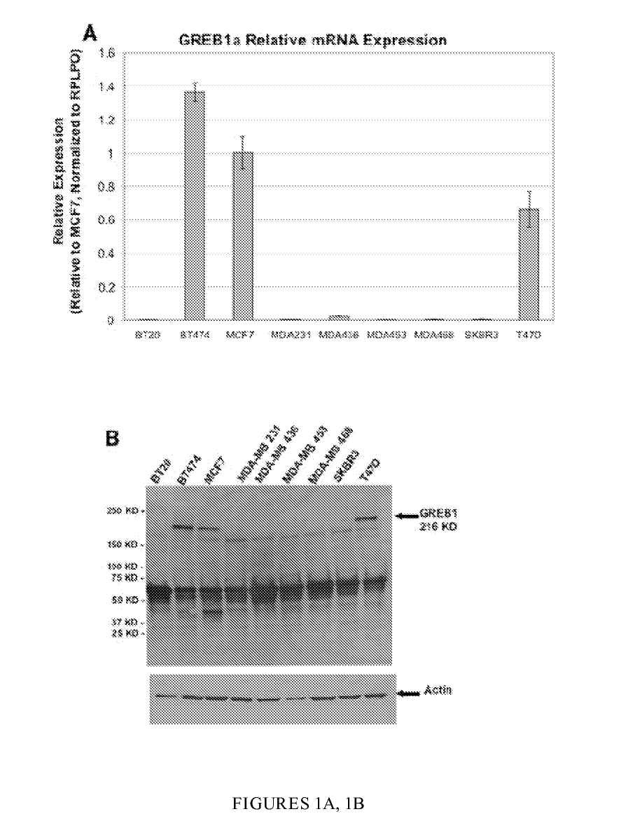

Correlation of GREB1 RNA and protein expression in ER⁺ and ER⁻ breast cancer cell lines: To investigate whether GREB1 RNA and protein expression levels were correlated in breast cancer cells, several ER⁺ and ER⁻ breast cancer cell lines were evaluated using RT-PCR and Western blot analysis. As observed in previous studies, ER⁺ breast cancer cell lines expressed detectable levels of GREB1 RNA by RT-PCR whereas ER⁺ breast cancer cell lines did not (FIG. 1A). Western blot analysis using the monoclonal GREB1ab and α-actin as a control revealed a similar pattern for the 216 kD GREB1 protein expression across these same cell lines (FIG. 1B). These results indicate that GREB1ab readily detects GREB1 protein expressed by ER⁺ breast cancer cell lines and that GREB1 RNA and protein expression profiles are comparable in these samples.

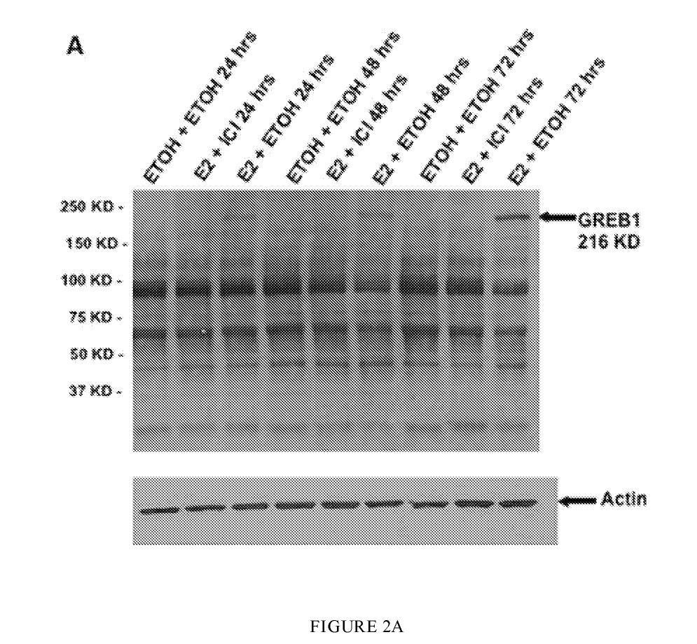

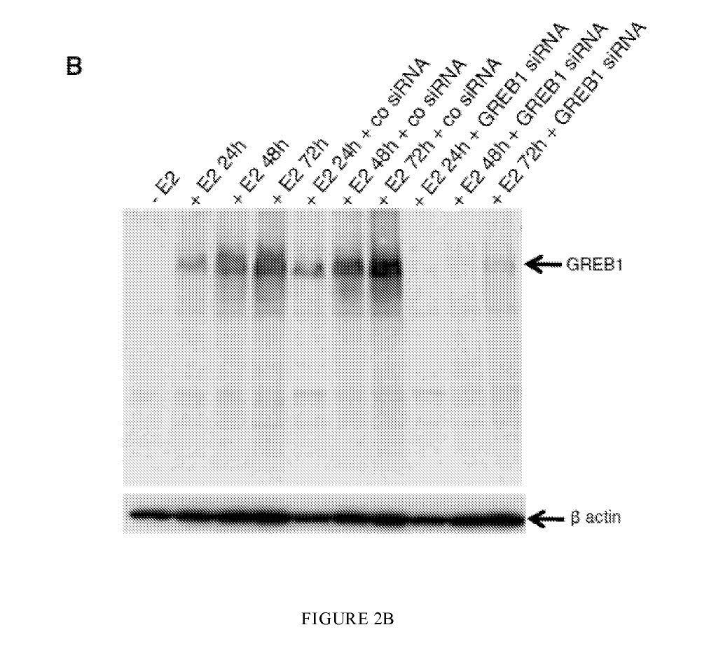

GREB1 RNA and protein expression in response to estrogen and estrogen inhibition: When ER⁺ breast cancer cells are cultured in the presence of estrogen, GREB1 mRNA expression is up-regulated as detected by RT-PCR. To determine if this enhanced expression was also observed with the GREB1 protein, lysates from ER⁺ MCF-7 cells grown in the presence or absence of estradiol, estrogen receptor antagonist ICI 182,780 (ICI) or the combination of estrogen with ICI were assayed for GREB1 protein expression by Western blot analysis (FIG. 2). A single band of 216 kD, identical to the expected molecular mass of full length GREB1, was observed in MCF-7 cells treated with estrogen for 24 and 48 hours while no protein was detected in MCF-7 cells grown under estrogen-free conditions. These results are consistent with real-time PCR results. Furthermore, GREB1 expression was reduced in MCF-7 cells treated with a combination of estradiol and ICI compared to cells treated with estrogen alone. Western blot analysis revealed silencing GREB1 mRNA expression using siRNA resulted in a loss of GREB1 protein expression up to 72 hours post-transfection compared to non-target control siRNA. Collectively, these data indicate that GREB1 protein levels respond as expected to estradiol stimulation and inhibition of estrogen receptor signaling and correlate with related mRNA expression profiles generated with real-time PCR.

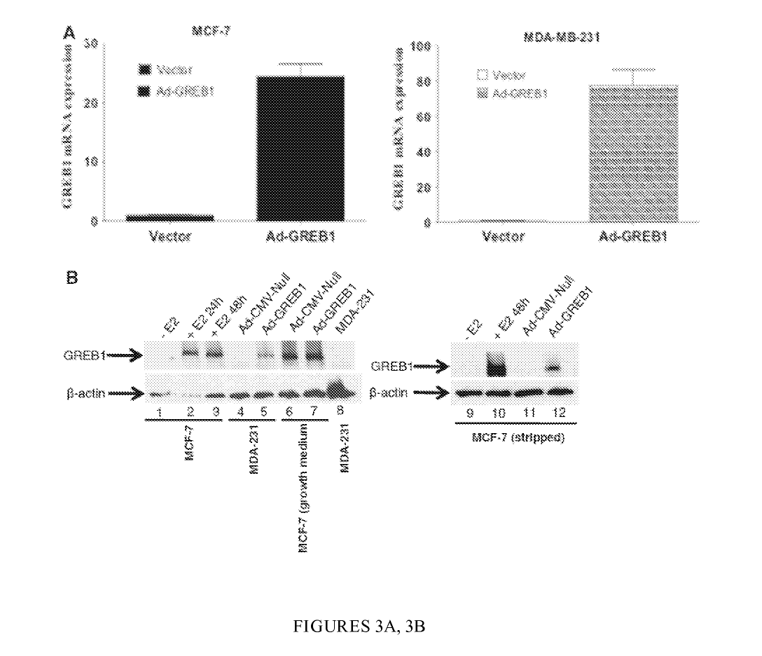

Detection of ectopic GREB1 protein expression by GREB1ab in an ER— breast cancer cell line: To further investigate the high specificity of the novel GREB1ab, GREB1 was overexpressed sing an adenoviral vector containing the full-length GREB1a cDNA in ER⁺ and ER⁻ breast cancer call lines (FIG. 3). GREB1⁻, ER⁻, MDA-MB-231 cells and 3-day estrogen depleted MCF-7 cells were infected with an adenoviral expression vector containing GREB1 mRNA (Ad-GREB1) or vector control (Ad-CMV-null) at an MOI of 20. Cells transduced with Ad-GREB1 expressed high GREB1 protein levels with the exogenous GREB1 protein sharing a mobility profile identical to that of endogenous GREB1 induced by estradiol stimulation (FIG. 3: lane 5 vs. lane 2 and 3; lane 12 vs. lane 10). The detectable expression of GREB1 in MCF-7 cells cultured in complete growth medium and transduced with Ad-CMV-null and Ad-GREB1 (FIG. 3: lane 6 vs. lane 7) was due to the existence of endogenous hormone in serum. GREB1 was undetectable in MCF-7 cells deprived of estrogen, parental MDA-MB-231 cells and MDA-MBA-231 cells transduced with empty vector (FIG. 3: lane 1, 9, 8 and 4). These results lend further evidence of the high specificity of this newly generated GREB1 antibody.

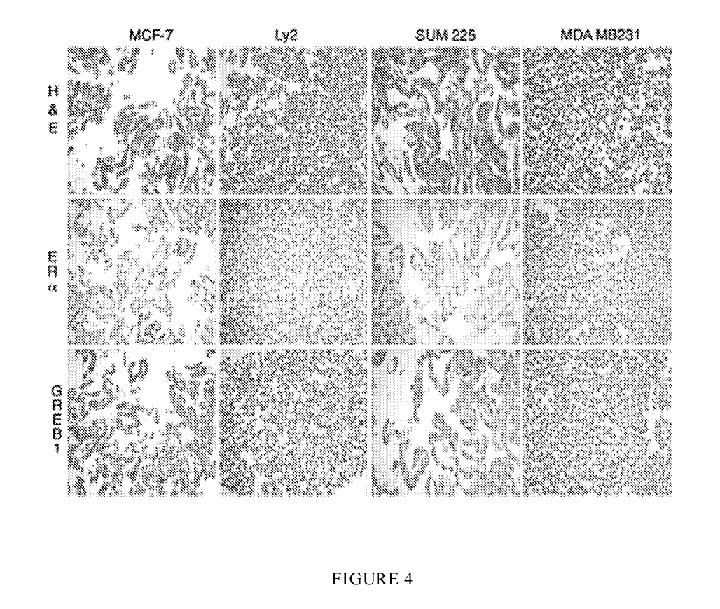

Immunohistochemistry of breast cancer cell lines using the monoclonal GREB1ab: Tissue microarrays generated from four breast cancer cell lines (MCF-7, Ly2, MDA-MB 231 and SUM 225) were processed and stained for immunohistochemical detection of GREB1 as well as ERα and HER2 (FIG. 4). GREB1 protein expression was detected in the ER⁺ breast cancer cell lines MCF-7 and Ly2 but not in ER-cell lines MDA-MB 231 or SUM 225. These results correlate with RT-PCR data that indicates GREB1 expression is elevated in ER⁺ breast cancer cells but undetectable in ER⁺ cells. This evidence suggests that GREB1 mRNA expression levels are directly related to GREB1 protein expression in ER⁺ and ER⁺ breast cancer cell lines.

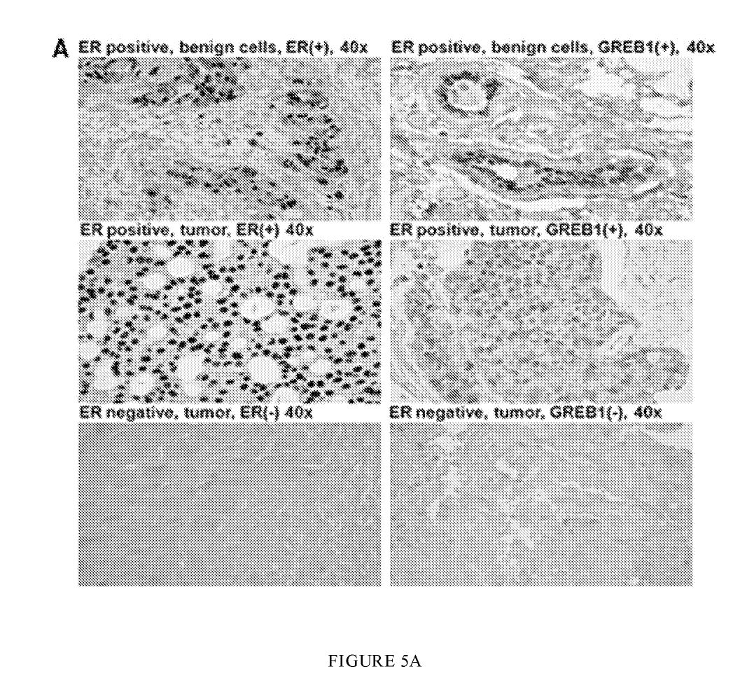

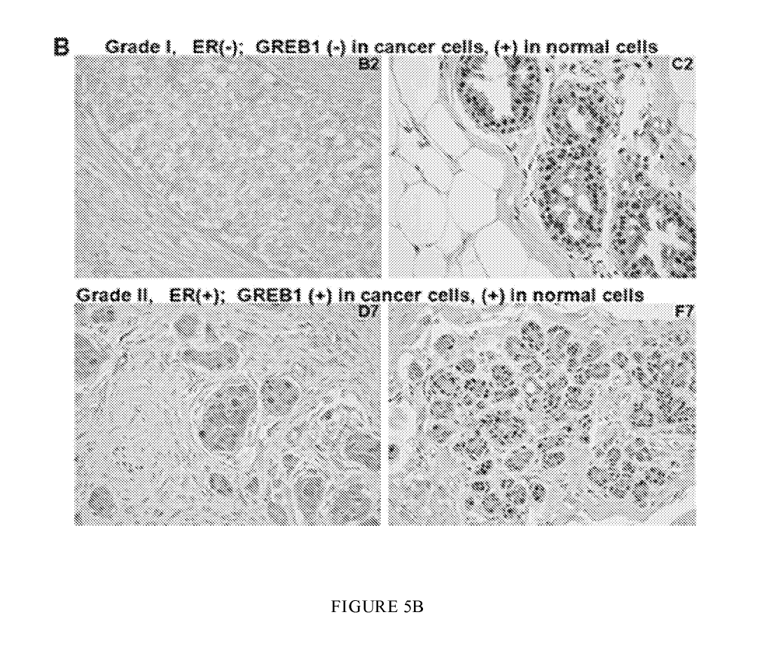

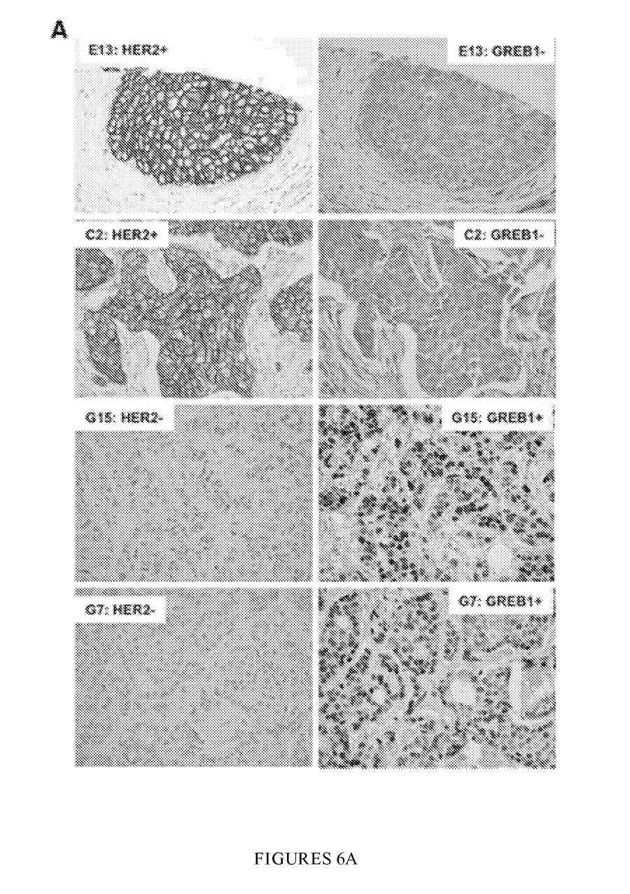

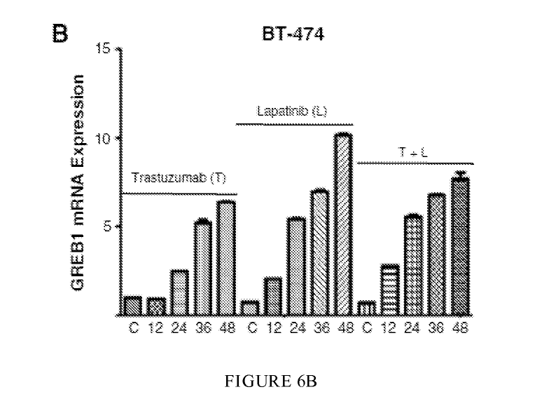

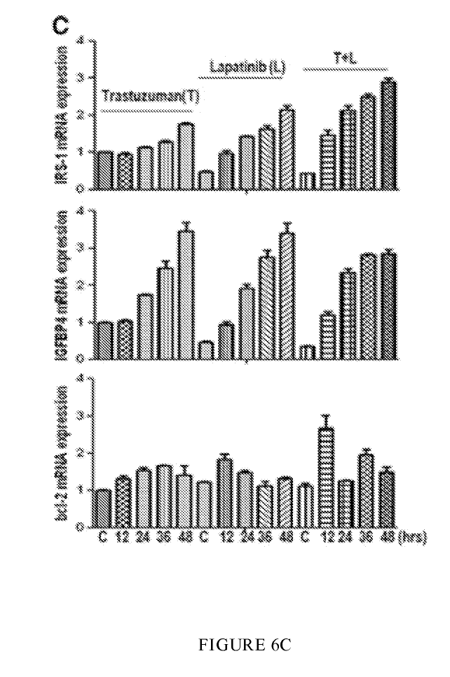

Immunohistochemical detection of GREB1 protein expression in ER⁺ and ER⁻ primary breast tumors: Using the GREB1ab, GREB1 protein expression was examined in breast cancer tissue sections from whole tumor blocks. GREB1 protein was detected in ER positive breast cancer tissue as well as normal breast tissue with little or no GREB1 expression in ER negative breast cancer tissue (FIG. 5A). Tissue microarrays of human breast cancer samples from Tissue Array Networks (Tissue-Array.Net) were also employed to further assess association between the GREB1 protein and ER status in primary cancers. 192 cases (105 ER′ and 87 ER⁻ cancers) provided assessable cores paired with corresponding uninvolved tissue from the same patient. This analysis showed a significant correlation between ER and GREB1 expression in primary breast cancers (Table 1, Phi correlation coefficient 0.5, P<0.0001). Representative micrographs from two tumors from the TMAs are presented in FIG. 5B. Panel B2 shows the absence of GREB1 staining in an ER-negative breast cancer whereas panel C2 reveals GREB1 staining in the normal mammary tissue adjacent to the B2 tumor sample. GREB1 protein was detected in both tumor (panel D7) and the paired, uninvolved normal tissue (panel F7) in an ER-positive breast cancer. These IHC experiments further correlate GREB1 protein expression in ER⁺ and ER⁻ breast tumors with previously reported GREB1 mRNA levels and provide a validated application for GREB1ab in staining primary breast cancer samples.

Inverse correlation between GREB1 and HER2 protein expression: Microarray and RT-PCR analysis have revealed an inverse correlation between GREB1 expression and HER2 status in human breast cancer. In addition, over-expression of HER2 in MCF-7 cells resulted in down-regulated GREB1 mRNA expression (Creighton C J, et al. (2006). Cancer Research 66(7):3903-3911). Breast cancer tissue whole sections and tissue microarrays were employed to verify this inverse correlation between GREB1 and HER2 at the protein level. HER2 status was determined in the above sections using previously described HER2 immunostaining diagnostic standards (FIG. 8A) (Wolff AC, et al. J Clin Oncol 25(1):118-145; Wolff AC, et al (2007) Archives of Pathology & Laboratory Medicine 131(1):18-43). The GREB1 gene expression inversely correlates with HER2 status (Table 2, Phi correlation coefficient 0.4, P<0.0001). More specifically, the correlation occurs only in ER-positive but not in ER negative tumors, most of which (87%) lack GREB1 expression. TRASTUZUMAB (a humanized monoclonal antibody directed at the extracellular domain of HER2) and LAPATINIB (a dual tyrosine kinase inhibitor that targets both EGFR and HER2) were used to test whether the lack of or decreased level of GREB1 expression in HER⁺, ER⁺ breast cancers is caused by the increased level of HER2. BT-474, which is a low ER positive, HER2 amplified breast cancer cell line, was found to express much lower GREB1 mRNA than MCF-7 cell line. Upon treatment of BT-474 with TRASTUZUMAB, LAPATINIB or the combination for 12, 24, 36 and 48 hours, GREB1 mRNA expression was increased (FIG. 8B). The pretreatment of BT-474 cells with TRASTUZUMAB increased GREB1 mRNA by 2.5 to 6.4 fold, which peaked at 48 hours. LAPATINIB enhanced GREB1 mRNA expression by 2 to 10 fold at 48 hours Inhibition of HER2 in BT474 cells results in increased ER expression so using the same samples as above, IRS-1, IGFBP4 and bcl-2 mRNA expression, three other canonical estrogen-regulated genes were examined next. The results show IGFBP4, IRS-1 and bcl-2 mRNAs are maximally enhanced by 3.5, 1.8, and 1.7 fold respectively by TRASTUZUMAB and by 3.4, 2.2 and 1.8 fold respectively with LAPATINIB (FIG. 8C), strongly suggesting HER2 likely exerts its effects on GREB1 by way of the estrogen receptor cGREB1 ade. This set of experiments incorporating application of the GREB1ab for IHC, real-time PCR and therapeutics verifies the inverse correlation of GREB1 and HER2 at the protein level as well as how inhibition of HER2 permits re-expression of ER and the ER signaling pathway as noted via GREB1 protein expression.

Epitopes within human GREB1 protein that may be used to generate monoclonal antibodies comprise:

| (residues #10-30; SEQ ID NO: 12) | |
|---|---|
| GREB1a#1: | KTT RFE EVL HNS IEA SLR SNN. |
| (residues #1109-1127; SEQ ID NO: 13) | |
| GREB1a#2: | SEK RSP MKR ERS RSH DS. |
| (residues #1915-1932; SEQ ID NO: 14) | |
| GREB1a#3: | RLE LED EWQ FRL RDE FQT. |

TABLE 1
Correlation of ER and GREB1 protein
expression in breast cancer patients

| TMA (total) | GREB1+ | GREB1− | Total |
|---|---|---|---|
| ER+ | 42 | 63 | 105 |
| ER− | 11 | 76 | 87 |
| Total | 53 | 139 | 192 |

Fisher exact probability test: P < 0.0001

TABLE 2
Correlation of HER2 and GREB1 protein expression
in ER+ breast cancer patients

| TMA (total) | GREB1+ | GREB1− | Total |
|---|---|---|---|
| HER2+ | 4 | 17 | 21 |
| HER2− | 38 | 46 | 84 |
| Total | 42 | 63 | 105 |

Chi-square: P < 0.0001

Discussion:

Characterizing the genes and gene products involved in estrogen-stimulated growth of breast tissue and cancer is necessary to understand oncogenesis as well as develop effective diagnostic assays and effective therapeutics. Based upon gene expression profiling studies completed with breast cancer cell lines, GREB1 was identified as one of the most sensitive gene products to estrogen induction or anti-estrogen therapies in ER⁺ breast cancers. These initial results suggested that GREB1 may have a potential role as a clinical marker for response to endocrine therapy. Subsequent siRNA silencing experiments targeting GREB1 mRNA expression revealed this gene product was required for estrogen-induced breast cancer cell proliferation and may be a potential candidate for new therapeutic strategies in breast cancer. At the very least, these initial studies involving analysis of GREB1 mRNA suggest a critical role for GREB1 in hormone-stimulated breast cancer progression and warrant further investigation for potential clinical applications.

Until now, all investigations of GREB1 and its role in estrogen-stimulated growth of breast cancer cells have focused on analysis of mRNA levels by microarray and PCR-based methods. The development of a novel monoclonal antibody that detects a single band of 216 kD by Western blotting that corresponds to the expected molecular mass of human GREB1 protein is described herein. The GREB1ab is validated for immunoblotting and immunohistochemical methods involving normal and diseased breast tissues as well as breast cancer cell lines. These methods and the GREB1ab were employed to verify that GREB1 protein is significantly expressed in ER-positive breast cancer cells and normal breast tissue with no GREB1 expression in ER⁻ samples as previously observed in mRNA-based studies. This observation provides a definitive correlation between GREB1 protein expression and ER status in primary breast cancers indicating that GREB1 may be a potential surrogate marker for ER. The GREB1ab was also employed to demonstrate that GREB1 protein is inversely correlated to HER2 status as inferred by early studies using RNA-based microarrays and RT-PCR. Thus, applications with this novel antibody suggest GREB1 protein level may predict both ER and HER2 expression in ER⁺ breast cancer cells. Most importantly, our findings provide definitive evidence that GREB1 mRNA levels are strongly correlated with protein expression levels in normal breast tissue and breast cancer cells. This is the first study describing the use of a monoclonal antibody targeting GREB1 and verifying that both mRNA and protein levels of this marker may have critical relevance to breast cancers.

To employ GREB1 as a biomarker in the management of breast cancer, further investigation is required to address the function and regulation of GREB1 in breast tissue and carcinogenesis. The knowledge that GREB1 mRNA expression profiles are directly reflected by the corresponding protein levels in normal breast and breast cancer cells expands the repertoire of technologies and methodologies that may be used to pursue these unknown parameters. The GREB1ab will be a valuable reagent for studies designed to elucidate the distribution and function of GREB1 protein in hormone-mediated cancers.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the disclosure will allow the reader to quickly GREB1 ertain the nature of the technical disclosure. It is submitted with the understanding that it will not be used to interpret or limit the scope or meaning of the following claims.

What is claimed is:

1. An isolated antibody which specifically binds to GREB1 proteins, polypeptides, peptides, nucleic acids, mutants, variants, isoforms, fragments or derivatives thereof, in vitro or in vivo.

2. The isolated antibody of claim 1, wherein the antibody is polyclonal or monoclonal.

3. The isolated antibody of claim 1, wherein the antibody specifically binds to animal or mammalian GREB1 molecules.

4. The isolated antibody of claim 1, wherein the antibody specifically binds to human GREB1 molecules.

5. The isolated antibody of claim 1, wherein the antibody specifically binds to epitopes comprising amino acid sequences set forth as SEQ ID NOS: 1, 12, 13, 14 or combinations thereof.

6. The isolated antibody of claim 1, further comprising a radiolabel.

7. A composition comprising an isolated antibody which specifically binds to GREB1 epitopes in vivo or in vitro.

8. The composition of claim 7, wherein the isolated antibody specifically binds to animal or mammalian GREB1 molecules.

9. The composition of claim 7, wherein the antibody specifically binds to human GREB1 molecules.

10. The isolated antibody of claim 7, wherein the antibody specifically binds to epitopes comprising amino acid sequences set forth as SEQ ID NOS: 1, 12, 13, 14 or combinations thereof.

11. A biomarker for the prognosis of disease progression and/or prediction of clinical outcome comprising at least one of: GREB1 proteins, polypeptides, peptides, nucleic acids, mutants, variants, isoforms, fragments or derivatives thereof.

12. The biomarker of claim 11, wherein increase in expression of GREB1 markers and decrease in expression of HER as compared to a normal control are prognostic for disease progression and/or prediction of clinical outcome.

13. A biomarker for diagnosis of cancer outcome comprising at least one of: GREB1 proteins, polypeptides, peptides, nucleic acids, mutants, variants, isoforms, fragments or derivatives thereof.

14. The biomarker of claim 13, wherein GREB1 expression is increased in estrogen receptor positive tumors as compared to HER and normal controls.

15. An isolated oligonucleotide for modulating expression or function of GREB1 comprising an antisense oligonucleotide comprising at least 5 consecutive nucleobases which are complementary to a sense or antisense polynucleotide of GREB1.

16. A method of treating cancer comprising:
administering to a patient in need thereof, a therapeutically effective amount of a GREB1 specific antibody or fragment thereof; and,
treating cancer.

17. The method of claim 16, wherein the GREB1 specific antibody or fragment thereof is optionally conjugated to one or more agents.

18. The method of claim 16, wherein the one or more agents comprise: chemotherapeutic agent, toxin, radioisotope, anti-angiogenic agent, cytokine or receptors thereof, cytostatic agents, or apoptosis inducing agents.

19. A kit comprising a GREB1 antibody, reagents and instructions for use thereof.

20. An aptamer specific for GREB1 molecules.
